(19) United States
(12) Patent Application Publication
(10) Pub. No.: US 2015/0038680 A1
(43) Pub. Date: Feb. 5, 2015

(54) SPIDER SILK DRAGLINE POLYNUCLEOTIDES, POLYPEPTIDES AND METHODS OF USE THEREOF

(71) Applicant: The Refents of the University of University of California, Oakland, CA (US)

(72) Inventors: Cheryl Y. Hayashi, Riverside, CA (US); Nadia A. Ayoub, Lexington, VA (US); James Starrett, Riverside, CA (US)

(73) Assignee: The Regents of the University of California, Oakland, CA (US)

(21) Appl. No.: 14/379,982

(22) PCT Filed: Feb. 21, 2013
(86) PCT No.: PCT/US13/27182

§ 371 Date: Aug. 20, 2014

Related U.S. Application Data
(60) Provisional application No. 61/601,320, filed on Feb. 21, 2012.

(51) Int. Cl. C07K 14/435

(52) U.S. Cl. CPC C07K 14/43518
USPC 530/353; 536/23.5; 435/320.1; 435/254.21; 435/348; 435/419; 435/69.1

(57) ABSTRACT

The disclosure provides spider silk polypeptides and polynucleotides encoding the same. Methods of using such polypeptides and polynucleotides and designing novel biomaterials using repeat units of the polypeptides and polynucleotides.

### CROSS REFERENCE TO RELATED APPLICATIONS

This application claims priority under 35 U.S.C. §119 from Provisional Application Ser. No. 61/601,320, filed Feb. 21, 2012, the disclosure of which is incorporated herein by reference.

### STATEMENT AS TO FEDERALLY SPONSORED RESEARCH

The U.S. Government has certain rights in this disclosure pursuant to Grant No. W911NF-06-1-0455 awarded by the Army Research Office and Grant No. DEB-0910365 awarded by the National Science Foundation.

### FIELD OF THE DISCLOSURE

The disclosure relates to spider silk dragline polypeptides, polynucleotides and uses thereof.

### BACKGROUND

Ever increasing demands for materials and fabrics that are both light-weight and flexible without compromising strength and durability has created a need for new fibers possessing higher tolerances for such properties as elasticity, denier, tensile strength and modulus. The search for a better fiber has led to the investigation of fibers produced in nature, some of which possess remarkable qualities.

Silk is vital to the ecology of spiders, being used throughout their lifetime for a wide array of essential purposes. There are over 42,000 described species of spiders (Platnick, 2011), and they are not only taxonomically diverse but also ecologically diverse in their silk biology. Yet few species have been sampled for their silk genes

### SUMMARY

The spider silk compositions provided herein find uses in the textile industry (e.g., as filaments, yarns, ropes, and woven material). Such materials made using the methods and compositions described herein will take advantage of the extreme toughness, tensile strength, and extensibility of silk. In addition, the polypeptides of the disclosure can be used in pliant energy absorbing devices including armor and bumpers. Besides the mechanical properties of spider silk, silk is proteinaceous (thus not petroleum-based like nylon or Kevlar®). Accordingly, the polypeptides of the disclosure provide biocompatible and biodegradable material useful in various industries including textiles and medicine. For example, the supercontraction ability of dragline silk can be beneficial for sutures that can tighten, compression bandages, or space minimizing packaging. Additionally the polypeptides can be used in the generation of scaffolds and material in tissue engineering, implants and other cell scaffold-based materials.

The disclosure provides a number of isolated spider silk polynucleotides. The polynucleotides encode the Spidroin proteins from various spider species.

The disclosure provides a substantially purified polypeptide comprising an N-terminus sequence that is about 80-150 amino acids in length and having at least about 90% identity to SEQ ID NO:5 from amino acid position 71 to 151 followed by a sequence of SEQ ID NO:5 from position 1358-1384, wherein SEQ ID NO:5 from position 1358-1384 is preceded and/or followed by a glycine and alanine rich region and having a C-terminal region of about 50-100 amino acids in length and having at least about 90% identity to SEQ ID NO:5 from position 2085 to 2141, wherein the polypeptide has a property selected from the group consisting of (i) a Young's modulus of about 3.94 GPa, (ii) an Ultimate Strength of about 139 MPa, (iii) an Extensibility of about 0.818 mm/mm, (iv) a Toughness of about 66.7 MPa and (v) any combination of (i)-(iv). In one embodiment, the polypeptide comprises a sequence that is at least 95% identical to a sequence selected from the group consisting of SEQ ID NO:3, 5, 73, 75, 77, 79, 81 and 83. In another embodiment, the polypeptide comprises a sequence selected from the group consisting of SEQ ID NO:3, 5, 73, 75, 77, 79, 81 and 83. In a specific embodiment, the polypeptide comprises SEQ ID NO:5. In yet a more specific embodiment, the polypeptide consists of SEQ ID NO:5.

The disclosure also provides a substantially purified polypeptide comprising an N-terminal region that is at least 60% identical to SEQ ID NO:7 from amino acid 1 to about 167, followed by a tandem array of 16-20 repeat units of about 200-370 amino acid in length and having a C-terminal domain comprising at least 40% identity to SEQ ID NO:7 from about amino acid 6236 to 6333, wherein the polypeptide has a property selected from the group consisting of (i) a Young's modulus of about 9.8 to 10.4 GPa, (ii) an Ultimate strength of about 636 to 687 MPa, (iii) an Extensibility of about 0.505 to 0.83 mm/mm, (iv) a Toughness of about 230 MPa to 376 MPa and (v) any combination of (i)-(iv). In one embodiment, the polypeptide is about 4400-6300 amino acids in length and about 430 to about 630 kiloDaltons in molecular weight. In another embodiment, the polypeptide is at least 90% identical to SEQ ID NO:7 or 87. In yet another embodiment, the polypeptide is at least 95% identical to SEQ ID NO:7 or 87. In yet a further embodiment, the polypeptide comprises SEQ ID NO:7. In yet another embodiment, the polypeptide comprises SEQ ID NO:87.

The disclosure also provides polynucleotides encoding any of the foregoing sequences. In another embodiment, the disclosure comprise a vector and/or host cell containing the polynucleotide.

The disclosure also provides a substantially purified polypeptide comprising a sequence selected from the group consisting of SEQ ID NO:3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107.

The disclosure also provides an isolated polynucleotide molecule selected from the group consisting of: (a) a polynucleotide molecule comprising a nucleotide sequence which is at least 80% identical to the nucleotide sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106; (b) a polynucleotide molecule which encodes a polypeptide comprising the amino acid sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107; (c) a polynucleotide molecule which encodes a naturally occurring allelic variant of a polypeptide comprising the amino acid sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107, wherein the polynucleotide molecule hybridizes to a polynucleotide molecule comprising SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106, or a complement thereof, under moderate to highly stringent conditions.

The disclosure also provides an isolated polynucleotide encoding a polypeptide comprising the amino acid sequence represented by SEQ ID NO: 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107 containing up to 30 conservative amino acid substitutions.

The disclosure provides an isolated polynucleotide comprising the nucleotide sequence represented by SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106.

The disclosure also provides a method for producing a silk dragline, the method comprising culturing the recombinant host cell of the disclosure under conditions suitable for expression of the polypeptide, such that the polypeptide is produced.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 shows a phylogeny for spider groups analyzed in the present disclosure. Phylogeny is based on Coddington and Levi (1991) and Hedin and Bond (2006).

FIG. 2A-B show (A) a schematic of alignment of Latrodectus hesperus Egg Case Proteins and Liphistius malayanus Egg Case Protein-like proteins. (B) An alignment of amino acids sequences, abbreviated using single letters. Only partial Latrodectus (Latr) ECPs are shown as Liphistius (Lipp) ECPLs lack the extended repetitive region. Alignment columns were highlighted using GeneDoc (Nicholas and Nicholas, 1997) according to physiochemical properties. Upper-case single letters occur above alignment positions showing 100% amino acid conservation, while lower case single letters occur above positions showing >50% conservation.

FIG. 3 shows a spidroin gene tree based on ML analysis of carboxy-terminal encoding region with gaps coded as binary characters and monophyly of some groups constrained. Numbers next to nodes and terminals correspond to numbers in tables S1 and S2 showing support values, alternate rootings, and continuous character data. Black circles indicate duplication events inferred by reconciliation. Branches are colored according to spidroin consensus repeat length using the combination of long repeat lengths for Hypochilus fib2 and Plectreurys fib3. Ancestral state parsimony optimization was determined by Mesquite v. 2.74. Bin ranges were determined by natural gaps in the distribution of repeat lengths for terminal and reconstructed internal nodes. Hash marks on branch indicate arbitrary shortening of branch for figure quality purposes. Brackets indicate clades with the following abbreviations: Me=Mesothelae, My=Mygalomorphae, Ar=Araneomorphae, AcSp=Aciniform, TuSp=Tubuliform, PySp=Pyriform, MaSp=Major ampullate, MiSp=Minor ampullate, Flag=Flagelliform.

FIG. 4 shows majority rule consensus of ensemble repeats within spidroins. Ensemble repeats are tandemly arrayed. Amino acid sequences with single letter abbreviations are shown. Alanine, serine, and glycine are highlighted. Single amino acids repeated in tandem are underlined.

FIG. 5 shows a heat map of percent compositions of alanine, glycine, and serine from spidroin repetitive regions. Cladogram adjacent to heat map shows relationships as in FIG. 3. Hexura fib1 was omitted since no repetitive region sequence was obtained for that cDNA. Histograms on columns also show relative composition levels of the three amino acids across spidroins. Abbreviations for Glade names are as in FIG. 3.

FIG. 6 shows an alignment of DNA sequences for Liphistius fib1 repeats. Amino acid translation and DNA consensus sequences are above repeat sequences. Dots indicate identity to the consensus sequence. Non-synonymous and synonymous differences from the consensus are indicated by upper and lower case letters, respectively.

FIG. 7 depicts a spidroin gene tree based on ML analysis of carboxy-terminal encoding region with gaps coded as binary characters and monophyly of some groups constrained. Numbers at nodes correspond to node numbers in supplementary tables 1 and 2. Node numbers indicated in red are constrained nodes. Green dots indicate nodes that do not conflict between the analysis with node constraints and the unconstrained ML analysis. Dots at terminal nodes indicate web type constructed by the taxa from which the spidroin sequence was obtained (red=trapdoor, blue=sheetweb, purple=purseweb, teal=turret). Hash marks on branch indicate arbitrary shortening of branch for figure quality purposes. Abbreviations for Glade names are as in FIG. 3.

FIG. 8 shows a schematic of the domains (N-terminal, repeat, and C-terminal) present in the particular sequences.

FIG. 9A-D shows the polyucleotide (SEQ ID NO:4) and polypeptide (SEQ ID NO:5) sequence of a silk protein of the disclosure.

FIG. 10A-G shows the polyucleotide (SEQ ID NO:6) and polypeptide (SEQ ID NO:7) sequence of a silk protein of the disclosure.

### DETAILED DESCRIPTION

As used herein and in the appended claims, the singular forms “a,” “and,” and “the” include plural referents unless the context clearly dictates otherwise. Thus, for example, reference to “protein” includes a plurality of such proteins and reference to “the peptide” includes reference to one or more peptides known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of:”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Spider silks have been demonstrated to have several desirable characteristics. The orb-web-spinning spiders can produce silk from six different types of glands. Each of the six fibers has different mechanical properties. However, they all have several features in common. They are (i) composed predominantly or completely of protein; (ii) undergo a transition from a soluble to an insoluble form that is virtually irreversible; and (iii) composed of amino acids dominated by alanine, serine, and glycine and have substantial quantities of other amino acids, such as glutamine, tyrosine, leucine, and valine. The spider dragline silk fiber has been proposed to consist of pseudocrystalline regions of antiparallel, 3-sheet structure interspersed with elastic amorphous segments.

The spider silks range from those displaying a tensile strength greater than steel (7.8 vs 3.4 G/denier) and those with elasticity greater than wool, to others characterized by energy-to-break limits that are greater than KEVLAR®. Given these characteristics spider silk could be used as a light-weight, high strength fiber for various textile applications.

Spider dragline silk has a number of unusual properties similar to other spider silks. These include a tensile strength greater than steel or carbon fibers (200 ksi), elasticity as great as some nylon (35%), a stiffness as low as silk (0.6 msi), and the ability to supercontract in water (up to 60% decrease in length). These properties are unmatched by any other material.

When spun into fibers, which can be done by dissolving spider silk in an appropriate solvent and forcing it through a small orifice, spider silk can have numerous uses. For example, one large volume use is for clothing. Silk with elasticity would have a unique place in the market even at high prices. It may also be applicable for certain kinds of high strength uses such as rope, surgical sutures, flexible tie downs for certain electrical components and even as a biomaterial for implantation (e.g., artificial ligaments or aortic banding). Thus, there are numerous applications including high-tech clothing, rope, sutures, medical coverings and others where various combinations of strength and elasticity are required. It is also possible to modify the properties of the silk fibers by altering the protein sequence.

However, even in view of the desirable uses, considerable difficulty has been encountered in attempting to solubilize and purify natural spider silk while retaining the molecular-weight integrity of the fiber. Spider silk fibers are insoluble except in very harsh agents such as LiSCN, LiClO₄, or 88% (vol/vol) formic acid. Once dissolved, the protein precipitates if dialyzed or if diluted with typical buffers. Another disadvantage of spider silk protein is that only small amounts are available from cultivated spiders, making commercially useful quantities of silk protein unattainable at a reasonable cost. Additionally, multiple forms of spider silks are produced simultaneously by any given spider. The resulting mixture has less application than a single isolated silk because the different spider-silk proteins have different properties and, due to solubilization problems, are not easily separated by methods based on their physical characteristics. Hence the prospect of producing commercial quantities of spider silk from natural sources has not previously been a practical one and there remains a need for an alternate mode of production. The technology of recombinant genetics provides one such mode.

By the use of recombinant molecular biology techniques it is now possible to transfer polynucleotides between different organisms for the purposes of expressing desired proteins in commercially useful quantities. Such transfer usually involves joining appropriate polynucleotides to a vector molecule, which is then introduced into a host cell or organism by transformation or transfection. Transformants are selected by a known marker on the vector, or by a genetic or biochemical screen to identify the cloned fragment. Vectors contain sequences that enable autonomous replication within the host cell, or allow integration into a chromosome in the host.

Progress has been made in the cloning and expression of spider silk proteins, Xu et al. (Proc. Natl, Acad. Sci. U.S.A., 87, 7120, (1990)) report the determination of the sequence for a portion of the repetitive sequence of a dragline silk protein from spider Nephila clavipes, based on a partial cDNA clone. The repeating unit is a maximum of 34 amino acids long and is not rigidly conserved. The repeat unit is composed of two different segments: (i) a 10 amino acid segment dominated by a polyalanine sequence of 5-7 residues; (ii) a 24 amino acid segment that is conserved in sequence but has deletions of multiples of 3 amino acids in many of the repeats. The latter sequence consists predominantly of GlyXₐₐGly motifs, with Xₐₐ being alanine, tyrosine, leucine, or glutamine (SEQ ID NO:1). The codon usage for this DNA is highly selective, avoiding the use of cytosine or guanine in the third position.

Hinman and Lewis (J. Biol. Chem. 267, 19320 (1992)) report the sequence of a partial cDNA clone encoding a portion of the repeating sequence of a second fibroin protein from dragline silk of Nephila clavipes. The repeating unit is a maximum of 51 amino acids long and is also not rigidly conserved.

The vast majority of spider silk knowledge is based on orb-weaving spiders. To extend the breadth of knowledge, silk cDNA transcripts and genes were characterized from non-orb-weaving spiders. Specifically, from Mesothelae (segmented spiders) and Mygalomorphae (e.g., tarantulas and trap door spiders). These sequences reported here belong to the spidroin and egg case protein gene families. Spidroins (contraction of “spider fibroin”) are the structural proteins that compose spider silk fibers. Egg case proteins are another type of structural proteins that, until now, were only known from the Latrodectus hesperus (Western black widow). The silk sequences provided herein provide insight as to how to designing silks using serine-rich motifs. By contrast, the widely studied major ampullate (dragline) silks of orb-weavers are glycine-rich. Mesothele and Mygalomorphae silks have desirable properties such as water resistance, microbial resistance, and able to persist in the environment for long periods of time.

While most silk research has been focused on derived members of Araneomorphae (“true spiders”), this disclosure provides silk genes from Paleocribelletae (a basal araneomorph Glade), increase sampling for Mygalomorphae (trapdoor spiders, tarantulas, and their kin; the sister group to Araneomorphae), and for the first time record silk sequences from Mesothelae (segmented spiders; the sister suborder to all other spiders) (Coddington and Levi, 1991). Mesotheles and Mygalomorphs exhibit profound differences in silk use compared to most araneomorph spiders (Coyle, 1986; Haupt, 2003). Mesotheles and Mygalomorphs produce general-purpose fibers and apply silk in a sheet-like manner to a burrow or other substrate, which is believed to be most similar to silk use in the common ancestor of extant spiders that lived >380 million years ago (Shear et al., 1989; Coddington and Levi, 1991; Haupt and Kovoor, 1993; Foelix, 1996; Vollrath and Selden, 2007; Ayoub et al., 2007a; Ayoub and Hayashi, 2009; Blackledge et al., 2009).

Full-length and extensive partial-length silk gene sequences were obtained from large-insert genomic DNA libraries for Latrodectus hesperus (Western Black Widow) and Argiope argentata (silver garden orb-weaver). The cDNA and genomic DNA derived sequences reported in this disclosure represent previously uncharacterized gene regions. Full length and near full-length sequences were determined for minor ampullate silk genes (web construction silk) and aciniform gland silk genes. The minor ampullate genes contain “spacer” regions, which are segments that disrupt the highly repetitive, alanine and glycine rich motifs that compose the bulk of the spidroin. Knowledge of the spacer sequences, their frequency, and spatial distribution within a silk protein are useful for successfully replicating minor ampullate silk. Also, the spacer sequences can be targeted for modification (such as for introducing tags or novel functional groups), while maintaining the tensile strength conferred by the surrounding glycine and alanine rich repetitive motifs. The aciniform silk sequences are the first complete gene sequences for one of the toughest spider silks ever characterized. Aciniform silk is used to wrap prey and to cushion the inside of egg sacs. Thus, these silk gene sequences provide key information for the design of biomaterials with desirable properties such as high toughness, resistance to degradation, being proteinaceous, and non-antigenic.

As used herein an “spidroin polypeptide” means a polypeptide that contains or comprises an amino acid sequence as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107; polypeptides having substantial homology or substantial identity to the sequences set forth in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107; polypeptides comprising from 1-50 (e.g., from 1-40, 1-30, 1-20, 1-15 or 1-10 conservative amino acid substitutions to any of the foregoing sequences; fragments of the foregoing sequences; and conservative and naturally occurring variants of the foregoing, wherein the polypeptide comprises a spider silk structure. The disclosure provides polypeptides having a sequence as set forth in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107 alone or fused to a second polypeptide.

A polypeptide of the disclosure encompasses an amino acid sequence that has a sufficient or a substantial degree of identity or similarity to a sequence set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107. Substantially identical sequences can be identified by those of skill in the art as having structural domains and/or having biological activity in common with Spidroin polypeptides. Methods of determining similarity or identity may employ computer algorithms such as, e.g., BLAST, FASTA, and the like.

Polypeptides of the disclosure may be modified by any type of alteration (e.g., insertions, deletions, or substitutions of amino acids; changes in the state of glycosylation of the polypeptide; refolding or isomerization to change its three-dimensional structure or self-association state; and changes to its association with other polypeptides or molecules) are also encompassed by the disclosure. Therefore, the polypeptides provided by the disclosure include polypeptides characterized by amino acid sequences similar to those as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107, but into which modifications are naturally provided or deliberately engineered. A polypeptide that shares biological activities in common with a polypeptide comprising a sequence as set forth in SEQ ID NOs: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107 having silk dragline characteristics or activity are encompassed by the disclosure.

The disclosure encompasses various forms of spidroin polypeptides that retain at least one activity or characteristic (“silk dragline characteristics”) selected from the group consisting (i) distinct structural units; and (ii) repeat units of a general structure comprising glycine and alanine rich regions separated by a spacer. For example, SEQ ID NO:5 comprises a full-length sequence comprising N-terminal and C-terminal regions that are optimzed to work with each other and with the repetitive regions therebetween. The repetitive region of minor ampullate silk is rich in glycine (G) and alanine (A), in regions of iterated A's (poly-A) and GA couplets. GGY and GQ motifs are also prevalent. These G and A rich regions are usually interrupted every-200-500 amino acids by a “spacer” region. The spacer regions of cob-web weavers are highly conserved in sequence and length. Adjacent −400 amino acid long G and A rich sequence and −28 amino acid long spacer form an ensemble repeat unit.

A typical structure for minor ampullate silk, taken from the middle of SEQ ID NO:5 is set forth below (corresponding to SEQ ID NO:5 from amino acid 1005-1384):

AASASGAVSTSAAAGYGQGAGTGAGGYGQGAGGYGQRAGAAAGGYGQGSG
GYGQGVGTAASVAAGGAGAGGYGLGAGGYGRGAGAAAGSGAGGYGQGAGG
YGQGAGAAAGAGAGGYGQGAGGYGQGAGAAASVAAGGAGAGGYGLGAGGY
GRGAGAGAGAAAGSGAGGYSQGAGGYGQRGGAAAGAAAGGAGSGGYGLGA
GIGAGAAAIAGGYGQGAGGYKQGAGAAAGAGTGAGGYGQGAGGYGQGAGA
AAGASAGAIAGGYGQGAVGYGQGTGGYGQGAAAGAGTGAGGYGQGAGGYG
QATGVGSYGQGTGDGARGPAGYGQGAGVGTIGAVGYGQGQTAGASSSTAA
GAASTGYTERQNEVTTTVSTTRKETADRRQ

Depicted is an alanine/glycine rich region followed by the spacer (the spacer region is underlined). The spacer region is highly conserved in sequence and length.

Thus, in one embodiment, the disclosure provides an isolated polypeptide comprising a sequence of SEQ ID NO:5 from position 1358-1384. For example, Scheme I, below, demonstrates the conservative nature of the spacer region across SEQ ID NOs: 3, 5, 73, 77 and 79.

| | |
|---|---|
| Consensus | SSTGYTERQNEVVTTVTTTRQETAD1RQ |
| | where 1 = Y/R |
| Seq 3 | SSTGYTERQNEVVTTVTTTRQETADYAQKQ |
| Seq 5 | SSTGYTERQNEVVTTVTTTRQEIADRRQ |
| Seq 5 | SSTGYTERQNEVVTTVTTTRQEIADRRQ |
| Seq 5 | ASTGYTERQNEVTTTVSTTRKETADRRQ |
| Seq 5 | SSTGYAGRQNEVITTVTTTRQETADYANKQ |
| Seq 73 | AGYTQRQNEVITTVSTTRQETADYGQKQ |
| Seq 73 | SGYTQKQNEVITTVSTTRQKIADYGQKQ |
| Seq 73 | SGYTQKQNEVITTVSTTRQKIADYGQKQ |
| Seq 73 | SGYTQRQNEVITTVSTTRQKTADYGQKQ |
| Seq 73 | SGYTQRQNEVITTVSTTRQKTADYGQKQ |
| Seq 73 | SGYIQKQNEVITTVSTTRQETADYGRKQ |
| Seq 77 | SSTGYTERQNEVVTTVTTTRQETADRRQ |
| Seq 77 | SSTGYTERQNEVVTSVTSTRQETADRRQ |
| Seq 77 | SSTGYTERQNEVITTVTTTRQETADQRQ |
| Seq 77 | SATGYTERQNEVVTTVTTTRQETADRRQ |
| Seq 77 | SSTGYTERQNEVVTTVTSTRQETADRRQ |
| Seq 77 | SATGYTERQNEVVTTVTTTRQETADRRQ |
| Seq 77 | SSTGYTERQNEVVTTVTSTRQETADRRQ |
| Seq 79 | SSTGYTERQNEVVTTVTTTRQESADYARKQ |

Scheme I, conserved nature of spacer sequences (multiple spacer are present in SEQ ID NOs: 5, 73, and 77).

In a further embodiment, the isolated polypeptide can comprise the sequence of SEQ ID NO:5 from position 1358-1384 and be preceded and/or followed by a glycine and alanine rich region. In yet still a further embodiment, the polypeptide can comprise an N-terminus sequence that is about 80-150 amino acids in length and having at least about 90% identity to SEQ ID NO:5 from amino acid position 71 to 151. The polypeptide can yet further comprise a C-terminal region of about 50-100 amino acids in length and having at least about 90% identity to SEQ ID NO:5 from position 2085 to 2141. For example, SEQ ID NOs: 3, 5, 73, 75, 77, 79, 81 and 83 contain all or parts of such structural motifs (see also FIG. 8). A polypeptide according to the foregoing will have biological characteristics including mechanical properties approximately the same or better than Latrodectus hesperus polypeptide of SEQ ID NO:5. For example, a polypeptide comprising a sequence of SEQ ID NO:5 from position 1358-1384 and be preceded and/or followed by a glycine and alanine rich region, having an N-terminus sequence that is about 80-150 amino acids in length and having at least about 90% identity to SEQ ID NO:5 from amino acid position 71 to 151 and a C-terminal region of about 50-100 amino acids in length and having at least about 90% identity to SEQ ID NO:5 from position 2085 to 2141, will have a Young's modulus of about 3.94 GPa, an Ultimate Strength of about 139 MPa, an Extensibility of about 0.818 mm/mm and a Toughness of about 66.7 MPa.

In yet another embodiment, the disclosure provides a silk polypeptide comprising a conserved N-terminal region of about 165 amino acids. Identical amino acids SEQ ID NO:7 and 87 are indicated in bold.

SEQ ID NO: 7
MNWLTSLSLIFILAFVQNVQVEGRKGHHHSSGSSKSPWANPAKANAFMKC
LIQKIS
SEQ ID NO: 87
MNWLPTLAFAILLL---SVQYDAVQSASTLS---RSPWANPAKAGSLMNC
LMSRIA
SEQ ID NO: 7
TSPVFPQQEKEDMEEIVETMMSAFSSMSTSGGSNAAKLQAMNMAFASSMA
ELVIAE
SEQ ID NO: 87
SSNVLPQQDKEDLESIMDTLMSAIKGASAKGKSSAAQLQAINMAVASSLA
EIVVAE
SEQ ID NO: 7
DADNPDSISIKTEALAKSLQQCFKSTLGSVNRHFIAEIKDLIGMFAREAA
AM-EEA
SEQ ID NO: 87
DAGNQASIAVKTQALTGALGQCFQAVMGTVDRKFINEINDLITMFAKEAA
SESNEI

The N-terminal region is followed by a repetitive region comprising extremely conserved repeats that are tandemly arrayed. In SEQ ID NO: 7 (from Western black widow), the repeat is about 370 amino acids long and there are 16 repeats in the repetitive region. In SEQ ID NO:87 (from the silver garden spider), the repeat is about 200 amino acids long and there are 20 repeats in the repetitive region. SEQ ID NO:7 and 87 can be characterized as (a) having repeat units that are hundreds of amino acids in length, (b) having extreme sequence conservation of repeats within a spidroins, at both the amino acid and nucleotide levels, (c) unlike most arthropod silks that are mostly Glycine, Alanine, and Serine, aciniform repeats contain numerous different amino acids, (d) the repeats are complex and cannot be explained by the few simple motifs common to other spider silks, such as poly-Alanine, Glycine-Alanine couplets, and (e) the immense length of the repetitive region makes aciniform spidroins the largest known spidroins (SEQ ID NO:7 has 6333 amino acids, 630 kiloDaltons predicted molecular weight; and SEQ ID NO:87 has 4479 amino acids and has a predicted molecular weight of 430 kiloDaltons). SEQ ID NO:87 repeat unit is almost half the length of the repeat length of SEQ ID NO:7 (200 vs. 370 amino acids). SEQ ID NO:87 repeat unit cannot be subdivided. However, the SEQ ID NO:7 repeat unit can be subdivided in half, a part A and a part B. Below is an exemplar repeat unit from SEQ ID NO:7. Non-underlined sequence corresponds to part A and underlined sequence corresponds to part B. The entire repeat unit (part A and part B) is tandem arrayed 16 times in the complete sequence.

FGGPSAGGDVAAKLARSLASTLASSGVFRAAFNSRVSTPVAVQLTDALVQ
KIASNLGLDYATASKLRKASQAVSKVRMGSDTNAYALAISSALAEVLLSS
GKVADANINQIAPQLASGIVLGVSTAAPQFGVDLSSINVNLDISNVARNM
QASIQGGPAPITAEGPDFGAGYPGGAPTDLSGLDMGAPSDGSRGGDATAK
LLQALVPALLKSDVFRAIYKRGTRKQVVQYVTNSALQQAASSLGLDASTI
SQLQTKATQALSSVSADSDSTAYAKAFGLAIAQVLGTSGQVNDANVNQIG
AKLATGILRGSSAVAPRLGIDLSGINVDSDIGSVTSLILSGSTLQMTIPA
GGDDLSGGYPGGFPAGAQPSGGAPVD
(Domain of SEQ ID NO: 7)

The repeat region is followed by a conserved C-terminal region. The conserved C-terminal region of the aciniform spidroins encoded by SEQ ID NOs:7 and 87 is about 100 amino acids long. Below is an alignment of the C-terminal regions. Identities are indicated by bold.

SEQ ID NO: 7
QGLKSPQASSRINRLSSSVVNALGPNGLDINNFSDGLRTTLSQLSSSGLS
SEQ ID NO: 87
VGLRSGSAASRIRQLTSSVTNAVGPNGVDANALARSLQSSFSNLRSSGMS
SEQ ID NO: 7
KKEAAIETLMEAMVALLQVLNSAQVNQVDTSSTVVTSSSLAKALSS-LF
SEQ ID NO: 87
SSDAKIEVLLETIVSLLQLLSNTQIRGVNPATASSVANSAARSFELVLA

Accordingly, the disclosure provides a polypeptide comprising an N-terminal region that is at least 60% identical to SEQ ID NO:7 from amino acid 1 to about 167, followed by a tandem array of 16-20 repeat units of about 200-370 amino acid in length and having a C-terminal domain comprising at least 40% identity to SEQ ID NO:7 from about amino acid 6236 to 6333. In a further embodiment, the polypeptide is about 4400-6300 amino acids in length and about 430 to about 630 kiloDaltons in molecular weight. The polypeptide has one or more silk-like characteristics including, but not limited to having a stress, strain and toughness approximately equal to the wild-type sequence isolated from Latrodectus Hesperus. For example, a polypeptide of the disclosure comprising an N-terminal region that is at least 60% identical to SEQ ID NO:7 from amino acid 1 to about 167, followed by a tandem array of 16-20 repeat units of about 200-370 amino acid in length and having a C-terminal domain comprising at least 40% identity to SEQ ID NO:7 from about amino acid 6236 to 6333 will have a Young's modulus of about 9.8 to 10.4 GPa, Ultimate strength of about 636 to 687 MPa, an Extensibility of about 0.505 to 0.83 mm/mm and a Toughness of about 230 MPa to 376 MPa.

As used herein a “unit repeat” constitutes a repetitive short or long sequence. Thus, in one embodiment a structural feature of the spider silk proteins can be considered to consist of a series of small variations of a unit repeat. The unit repeats in the naturally occurring proteins are often distinct from each other. That is, there is little or no exact duplication of the unit repeats along the length of the protein. Synthetic spider silks, however, can be made wherein the primary structure of the protein comprises a number of exact repetitions of a single unit repeat. Additional synthetic spider silks can be synthesized which comprise a number of repetitions of one unit repeat together with a number of repetitions of a second unit repeat. Such a structure would be similar to a typical block copolymer. Unit repeats of several different sequences can also be combined to provide a synthetic spider silk protein having properties suited to a particular application. The term “direct repeat” as used herein is a repeat in tandem (head-to-tail arrangement) with a similar repeat that may be directly adjacent of separated by 5-10 amino acids.

The disclosure provides partial, full-length and mature forms spidroin polypeptides. The polypeptide and polynucleotides of the disclosure were identified from various spider families as set forth below. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides may be encoded by a single genetic locus if multiple mRNA forms are produced from that locus by alternative splicing or by the use of multiple translation initiation sites. The “mature form” of a polypeptide refers to a polypeptide that has undergone post-translational processing steps, if any, such as, for example, cleavage of the signal sequence or proteolytic cleavage to remove a prodomain. Multiple mature forms of a particular full-length polypeptide may be produced, for example, by imprecise cleavage of the signal sequence, or by differential regulation of proteases that cleave the polypeptide. The mature form(s) of such polypeptide may be obtained by expression, in a suitable insect or mammalian cell or other host cell, of a polynucleotide that encodes the full-length polypeptide. The sequence of the mature form of the polypeptide may also be determinable from the amino acid sequence of the full-length form, through identification of signal sequences or protease cleavage sites. The spidroin polypeptides of the disclosure also include polypeptides that result from post-transcriptional or post-translational processing events such as alternate mRNA processing which can yield a truncated but biologically active polypeptide. Also encompassed within the disclosure are variations attributable to proteolysis such as differences in the N- or C-termini upon expression in different types of host cells, due to proteolytic removal of one or more terminal amino acids from the polypeptide (generally from 1-5 terminal amino acids).

A polypeptide of the disclosure may be prepared by culturing transformed or recombinant host cells under culture conditions suitable to express a polypeptide of the disclosure. The resulting expressed polypeptide may then be purified from such culture using known purification processes. The purification of the polypeptide may also include an affinity column containing agents which will bind to the polypeptide; one or more column steps over such affinity resins as concanavalin A-agarose, Heparin-Toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, the polypeptide of the disclosure may also be expressed in a form that will facilitate purification. For example, it may be expressed as a fusion polypeptide, such as those of maltose binding polypeptide (MBP), glutathione-5-transferase (GST) or thioredoxin (TRX). Kits for expression and purification of such fusion polypeptides are commercially available from New England BioLab (Beverly, Mass.), Pharmacia (Piscataway, N.J.), and Invitrogen, respectively. The polypeptide can also be tagged with an epitope and subsequently purified by using a specific antibody directed to such epitope. Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous recombinant polypeptide. The polypeptide thus purified is substantially free of other insect, plant, bacterial or mammalian polypeptides and is defined in accordance with the disclosure as a “substantially purified polypeptide”; such purified polypeptides include fragment, variant, and the like. A polypeptide of the disclosure may also be expressed as a product of transgenic animals or insects, which are characterized by somatic or germ cells containing a polynucleotide encoding a polypeptide of the disclosure.

It is also possible to utilize an affinity column such as a monoclonal antibody generated against polypeptides of the disclosure, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the disclosure. In this aspect of the disclosure, proteins that bind a polypeptide of the disclosure (e.g., an antibody of the disclosure) can be bound to a solid phase support or a similar substrate suitable for identifying, separating, or purifying cells that express polypeptides of the disclosure on their surface. Adherence of, for example, an antibody of the disclosure to a solid phase surface can be accomplished by any means, for example, magnetic microspheres can be coated with these polypeptide-binding proteins and held in the incubation vessel through a magnetic field.

A polypeptide of the disclosure may also be produced by known conventional chemical synthesis. Methods for constructing the polypeptides of the disclosure by synthetic means are known to those skilled in the art. The synthetically-constructed polypeptide sequences, by virtue of sharing primary, secondary or tertiary structural and/or conformational characteristics with native polypeptides may possess biological properties in common therewith, including biological activity.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Typically, the polypeptide of the disclosure is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Species homologues of spidroin polypeptides and polynucleotides of the disclosure encoding the polypeptides are also provided by the disclosure. As used herein, a “species homologue” is a polypeptide or polynucleotide with a different species of origin from that of a given polypeptide or polynucleotide, but with significant sequence similarity to the given polypeptide or polynucleotide. Species homologues may be isolated and identified by making suitable probes or primers from polynucleotides encoding the polypeptides provided herein and screening a suitable nucleic acid source from the desired species. Alternatively, homologues may be identified by screening a genome database containing sequences from one or more species utilizing a sequence (e.g., nucleic acid or amino acid sequence) of a spidroin polypeptide of the disclosure. Such genome databases are readily available for a number of species (e.g., on the world wide web (www) at tigr.org/tdb; genetics.wisc.edu; stanford.edu/.about.ball; hiv-web.lanl.gov; ncbi.nlm.nig.gov; ebi.ac.uk; and pasteur.fr/other/biology).

Intermediate Sequence Search (ISS) can be used to identify closely related as well as distant homologs by connecting two proteins through one or more intermediate sequences. ISS repetitively uses the results of the previous query as new search seeds. Saturated BLAST is a package that performs ISS. Starting with a protein sequence, Saturated BLAST runs a BLAST search and identifies representative sequences for the next generation of searches. The procedure is run until convergence or until some predefined criteria are met. Saturated BLAST is available on the world wide web (www) at: bioinformatics.burnham-inst.org/xblast (see also, Li et al. Bioinformatics 16(12): 1105, 2000).

Fragments of the Spidroin polypeptides of the disclosure are encompassed by the disclosure. Peptide fragments of Spidroin polypeptides of the disclosure, and polynucleotides encoding such fragments include amino acid or nucleotide sequence lengths that are at least 25% (typically at least 50%, 60%, or 70%, and commonly at least 80%) of the length of a spidroin polypeptide or polynucleotide of the disclosure. Typically such sequences will have at least 60% sequence identity (more typically at least 70%-75%, 80%-85%, 90%-95%, at least 97.5%, or at least 99%, and most commonly at least 99.5%) with spidroin polypeptide or polynucleotide of the disclosure when aligned so as to maximize overlap and identity while minimizing sequence gaps. Also included in the disclosure are polypeptides, peptide fragments, and polynucleotides encoding them, that contain or encode a segment comprising at least 8 to 10, typically at least 20, at least 30, or most commonly at least 40 contiguous amino acids. Such polypeptides and fragments may also contain a segment that shares at least 70% (at least 75%, 80%-85%, 90%-95%, at least 97.5%, or at least 99%, and commonly at least 99.5%) with any such segment of any of the Spidroin family of polypeptides, when aligned so as to maximize overlap and identity while minimizing sequence gaps. Visual inspection, mathematical calculation, or computer algorithms can determine the percent identity.

Certain spidroin polypeptides of the disclosure are composed of a small suite of amino acid sequence motifs, such as GGX and poly-A, which are repeated many times throughout the polypeptide. The amino acid sequence of a fragment these spidroins is repetitive and rich in glycine, but is otherwise unlike any previously known amino acid sequence. Consensus repeat domains comprise:

| | |
|---|---|
| GGYGQGAGGYGQGAGXaa₁A | (SEQ ID NO: 108) |

where Xaa1 is S or A residues.

The polypeptides of the disclosure can be made by direct synthesis or by expression from cloned polynucleotide of the disclosure. Means for expressing cloned polynucleotides are described herein and are generally known in the art. The following considerations are recommended for the design of expression vectors used to express polynucleotides encoding the spider silk polypeptides of the disclosure.

Because spider silk proteins are highly repetitive in their structure, cloned polynucleotides should be propagated and expressed in host cell strains that can maintain repetitive sequences in extrachromosomal elements. The prevalence of specific amino acids (e.g., alanine, glycine, proline, and glutamine) also suggests that it might be advantageous to use a host cell that overexpresses tRNA for these amino acids.

The proteins of the disclosure can otherwise be expressed using vectors (described more fully elsewhere herein) providing for high level transcription, fusion proteins allowing affinity purification through an epitope tag, and the like. The hosts can be either bacterial or eukaryotic or plant cells. Eukaryotic cells such as yeast, especially Saccharomyces cerevisisae, or insect cells might be particularly useful eukaryotic hosts. Expression of an engineered minor ampullate silk protein is described in U.S. Pat. No. 5,756,677, incorporated by reference herein. Such an approach can be used to express proteins of the disclosure.

In one aspect, a spidroin polypeptide or any combination thereof may be expressed in a plant cell. For example, crop plants can be engineered to express spider silk genes. In one embodiment, standard molecular biology techniques are used to generate transgenes that are transformed into a suitable plant host cell. The transgene constructs can comprise (from 5′ to 3′) the cauliflower mosaic virus promoter, signal peptide, silk protein-coding region, together with a 6Xhis tag (for detection with His antibody and protein purification) and KDEL signal (to assure retention in the ER) at the carboxy-termini. The chimeric silk protein construct will be inserted into the vector pMDC32. This vector will be used in Agrobacterium-mediated transformation of crop plants such as tobacco and tomato.

Alternatively, plastid transformation is an effective mechanism to over-produce recombinant proteins in plants. One advantage of plastid transformation is the fact that plastids are not found in most pollen grains and therefore there is a limited capacity for transgene flow to related weeds or crops. More importantly, a wide array of proteins from animals, plants and microbes have been expressed to high levels in plant chloroplasts with protein levels ranging from 0.6% to 31%. This high level of protein accumulation is attributed to the approximate 10,000 plastid genomes present per plant mesophyll cell. In addition, there are examples where protein accumulation is toxic in the cytosol or vesicles, but was non-toxic when the protein accumulated in the chloroplast. To date, tobacco plastid transformation is almost routine and tomato plastid transformation is now feasible. In one embodiment polynucleotides of the disclosure are introduced into plastids using the pRB94 vector. The coding region will be expressed from the strong Prrn protomer. The chimeric silk transgenes will be introduced into plant leaf segments using particle bombardment. Solanum lycopersicum cvMoney maker and Nicotiana tabacum Xanthi can be used as parent plants. Spectinomycin-resistant tomato callus transformants can be selected for and serially propagated. For example, while one transgenic chloroplast tomato line is recovered after bombardment of 10 plates, 14 transgenic tobacco homoplasmic lines are recovered. With each transfer to new media, transgenic calli will be screened using genomic DNA digests and DNA blots to detect parental and transgenic genomes. Homoplasmic lines (lines containing only the transgenic genomes) will be further characterized. The accumulation of silk proteins in the homoplasmic lines will be determined by immunoblots blots using His-tag antiserum and/or antibodies specific to peptides or polypeptides in the recombinant protein.

The levels of silk protein achieved in tobacco or tomato chloroplasts may partially be based on the codon usage in plant chloroplasts vs. spiders. There is a good correlation of codon usage in the silk protein RNA and codons utilized in tobacco chloroplasts.

A useful spider silk protein or fragment thereof may be (1) insoluble inside a cell in which it is expressed or (2) capable of being formed into an insoluble fiber under normal conditions by which fibers are made. Typically, the protein is insoluble under conditions (1) and (2). Specifically, the protein or fragment may be insoluble in a solvent such as water, alcohol (methanol, ethanol, etc.), acetone and/or organic acids, etc. The Spidroin polypeptides or fragment thereof should be capable of being formed into a fiber having high tensile strength. A fragment or variant may have substantially the same characteristics as a natural spider silk. The natural protein may be particularly insoluble when in fiber form and resistant to degradation by most enzymes.

Recombinant spider silk proteins may be recovered from cultures by lysing cells to release spider silk proteins expressed therein. Initially, cell debris can be separated by centrifugation. Clarified cell lysate comprised of debris and supernatant can then be repeatedly extracted with solvents in which spidroin polypeptides are insoluble, but cellular debris is soluble. These procedures can be repeated and combined with other procedures including filtration, dialysis and/or chromatography to obtain a pure product.

Fibrillar aggregates will form from solutions by spontaneous self-assembly of spider silk proteins when the protein concentration exceeds a critical value. The aggregates can be gathered and mechanically spun into macroscopic fibers. For example, the spider silk polypeptides can be viewed as derivatized polyamides. Accordingly, methods for producing fiber from soluble spider silk proteins are similar to those used to produce typical polyamide fibers, e.g. nylons, and the like. In one aspect, a spidroin polypeptide or combinations of spidroin polypeptides can be solubilized in a strongly polar solvent. The protein concentration of such a protein solution should typically be greater than 5% and is typically between 8 and 20%.

Fibers are spun from solutions having properties characteristic of a liquid crystal phase. The fiber concentration at which phase transition can occur is dependent on the polypeptide composition of a protein or combination of proteins present in the solution. Phase transition, however, can be detected by monitoring the clarity and birefringence of the solution. Onset of a liquid crystal phase can be detected when the solution acquires a translucent appearance and registers birefringence when viewed through crossed polarizing filters.

The solvent used to dissolve spidroin polypeptide should be polar. Such solvents are exemplified by di- and tri-haloacetic acids, and haloalcohols (e.g. hexafluoroisopropanol). In some instances, co-solvents such as acetone are useful. Solutions of chaotropic agents, such as lithium thiocyanate, guanidine thiocyanate or urea can also be used.

In one fiber-forming technique, fibers can first be extruded from the protein solution through an orifice into methanol, until a length sufficient to be picked up by a mechanical means is produced. Then a fiber can be pulled by such mechanical means through a methanol solution, collected, and dried.

As mentioned above, the spidroin polypeptides of the disclosure have primary structures that comprise repeating units. Synthetic spider silks can be generated wherein the primary structure of a synthetic spider silk protein can be described as a number of exact repetitions of a single unit repeat. Such a structure would be similar to a typical block copolymer. The disclosure also encompasses generation of synthetic spider silk proteins comprising unit repeats derived from several different spider silk sequences (naturally occurring variants or genetically engineered variants thereof).

Experiments on recombinant silks made with and without the C-terminal region showed that the C-terminus was useful for fibroins to form aggregates. Protein aggregation is an essential step in the precipitation of liquid spinning dope into a solid silk fiber. The C-terminus is useful for aggregation of recombinant fibroins, and for the formation of the characteristic crystalline structures that impart strength to silk fibers. As has been proposed for the C-terminus, the evolutionary conservation of the N-terminus suggests that this region is also functionally significant. For example, N-termini may play a central role in the proper transport of fibroins from secretory cells to silk gland lumen, aid in fiber formation, and contribute to the structural properties of silk fibers. Another evolutionarily conserved aspect of spider fibroins is their extremely large size, which is also a feature of independently evolved insect fibroins. Thus, large size has been repeatedly selected for in the evolution of fibroin genes. Therefore, a complete silk gene, with full representation of the N- and C-terminal regions, the intervening repetitive sequence, and the transitions among these domains, should dramatically improve recombinant silk performance.

In another aspect of the disclosure, a polypeptide may comprise various combinations of fibroin polypeptide domains. Also included are recombinant polypeptides and the polynucleotides encoding the polypeptides wherein the recombinant polypeptides are “chimeric polypeptides” or “fusion polypeptides” and comprise a sequence as set forth, for example, in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107, operatively linked to a second polypeptide. The second polypeptide can be any polypeptide of interest having an activity or function independent of, or related to, the function of the spidroin polypeptide. For example, the second polypeptide can be a domain of a related but distinct member of the fibroin family of polypeptides. The term “operatively linked” is intended to indicate that the spidroin sequence and the second polypeptide sequence are fused in-frame to each other. The second polypeptide can be fused to the N-terminus or C-terminus of spidroin sequence. Such fusion polypeptides can facilitate the purification of recombinant Spidroin polypeptides. In another embodiment, the fusion polypeptide comprises a Spidroin sequence comprising a heterologous signal sequence at its N-terminus.

The spidroin polypeptides of the disclosure can also include a localization sequence to direct the polypeptide to particular cellular sites by fusion to appropriate organellar targeting signals or localized host proteins. A polynucleotide encoding a localization sequence, or signal sequence, can be ligated or fused at the 5′ terminus of a polynucleotide encoding a spidroin polypeptide such that the signal peptide is located at the amino terminal end of the resulting fusion polynucleotide/polypeptide. In eukaryotes, the signal peptide functions to transport a polypeptide across the endoplasmic reticulum. The secretory protein is then transported through the Golgi apparatus, into secretory vesicles and into the extracellular space or the external environment. Signal peptides include pre-pro peptides that contain a proteolytic enzyme recognition site.

The localization sequence can be a nuclear-, an endoplasmic reticulum-, a peroxisome-, or a mitochondrial-localization sequence, or a localized protein. Localization sequences can be targeting sequences that are described, for example, in “Protein Targeting”, chapter 35 of Stryer, L., Biochemistry (4th ed.). W.H. Freeman, 1995. Some important localization sequences include those targeting the nucleus, mitochondria, endoplasmic reticulum, peroxisome (SKF), plasma membrane, CC, CXC and the like, cytoplasmic side of plasma membrane (fusion to SNAP-25), or the Golgi apparatus (fusion to furin).

A chimeric or fusion polypeptide of the disclosure can be produced by standard recombinant molecular biology techniques. In one embodiment, polynucleotide fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Examples of polynucleotides encoding all or portions of the Spidroin polypeptides are set for in SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide).

The disclosure further includes polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the disclosure in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase.

Additional variants within the scope of the disclosure include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide.

The disclosure also provides polynucleotides encoding spidroin polypeptides. The term “polynucleotide” refers to a polymeric form of nucleotides of at least 10 bases in length. The nucleotides can be ribonucleotides, deoxyribonucleotides, or modified forms of either type of nucleotide. The term includes single and double stranded forms of DNA or RNA. DNA includes, for example, cDNA, genomic DNA, chemically synthesized DNA, DNA amplified by PCR, and combinations thereof. The polynucleotides of the disclosure include full-length genes and cDNA molecules as well as a combination of fragments thereof. The polynucleotides of the disclosure are preferentially derived from human sources, but the disclosure includes those derived from non-human species, as well.

A polynucleotide of the disclosure will generally contain phosphodiester bonds, although in some cases, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Other analogs include peptide nucleic acids (PNA) which are peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

As described above, the nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant polynucleotide molecule, which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

A spidroin polynucleotide of the disclosure (1) encodes a polypeptide comprising a sequence as set forth in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107; (2) has a sequence as set forth in SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106; (3) has sequences complementary to a sequence as set forth in SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106; (4) fragments of SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106 or their complements that specifically hybridize to the polynucleotide of (2) or (3) under moderate to highly stringent conditions; and (5) polynucleotides of (1), (2), (3), or (4) wherein T can also be U (e.g., RNA sequences).

Among the uses of the disclosed spidroin polynucleotides, and combinations of fragments thereof, is the use of fragments as probes or primers. Such fragments generally comprise at least about 17 contiguous nucleotides of a DNA sequence. In other embodiments, a DNA fragment comprises at least 30, or at least 60 contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries.

The disclosure also includes polynucleotides and oligonucleotides that hybridize under reduced stringency conditions, typically moderately stringent conditions, and commonly highly stringent conditions, to spidroin polynucleotides described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, F. M. Ausubel et al., eds., John Wiley & Sons, Inc., sections 2.10 and 6.3-6.4, incorporated herein by reference), and can be readily determined by those having ordinary skill in the art based on, for example, the length and/or base composition of the polynucleotide. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS, SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target polynucleotide of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids above 18 base pairs in length, Tₘ (° C.)=81.5+16.6(log₁₀ [Na⁺])+0.41(% G+C)−(600/N), where N is the number of bases in the hybrid, and [Na⁺] is the concentration of sodium ions in the hybridization buffer ([Na⁺] for 1×SSC=0.165M). Preferably, each such hybridizing nucleic acid has a length that is at least 25% (more preferably at least 50%, 60%, or 70%, and most preferably at least 80%) of the length of a polynucleotide of the disclosure to which it hybridizes, and has at least 60% sequence identity (more preferably at least 70%, 75%, 80%, 85%, 90%, 95%, 97.5%, or at least 99%, and most preferably at least 99.50) with a polynucleotide of the disclosure to which it hybridizes.

“Conservatively modified variants” applies to both polypeptide and polynucleotide. With respect to particular polynucleotide, conservatively modified variants refer to codons in the polynucleotide which encode identical or essentially identical amino acids. Because of the degeneracy of the genetic code, a large number of functionally identical polynucleotides encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such variations are “silent variations,” which are one species of conservatively modified variations. Every polynucleotide sequence herein that encodes a polypeptide also describes every possible silent variation of the nucleic acid. One of skill will recognize that each codon in a polynucleotide (except AUG, which is ordinarily the only codon for methionine) can be modified to yield a functionally identical molecule. Accordingly, each silent variation of a nucleic acid that encodes a polypeptide is implicit in each described sequence.

The disclosure also provides methodology for analysis of polynucleotides of the disclosure on “DNA chips” as described in Hacia et al., Nature Genetics, 14:441-447 (1996). For example, high-density arrays of oligonucleotides consisting of a sequence as set forth in SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106, or a variant or mutant thereof are applied and immobilized to the chip and can be used to detect sequence variations in a population. Polynucleotides in a test sample are hybridized to the immobilized oligonucleotides. The hybridization profile of the target polynucleotide to the immobilized probe is quantitated and compared to a reference profile. The resulting genetic information can be used in molecular identification. The density of oligonucleotides on DNA chips can be modified as needed.

The disclosure also provides genes corresponding to the polynucleotides disclosed herein. “Corresponding genes” are the regions of the genome that are transcribed to produce the mRNAs from which cDNA molecules are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials.

Expression, isolation, and purification of the polypeptides and fragments of the disclosure can be accomplished by any suitable technique, including but not limited to the following methods and those described elsewhere herein.

The isolated polynucleotides of the disclosure may be operably linked to an expression control sequence such as the pMT2 or pED expression vectors disclosed in Kaufman et al., Nucleic Acids Res. 19:4485 (1991); and Pouwels et al. Cloning Vectors: A Laboratory Manual, Elsevier, New York, (1985, and Supplements), in order to produce a polypeptide of the disclosure recombinantly. Many suitable expression control sequences are known in the art. General methods of expressing recombinant polypeptides are also known and are exemplified in R. Kaufman, Methods in Enzymology 185:537 (1990). As defined herein “operably linked” means that an isolated polynucleotide of the disclosure and an expression control sequence are situated within a vector or cell in such a way that the polypeptide encoded by the polynucleotide is expressed by a host cell which has been transformed (transfected) with the vector or polynucleotide operably linked to the control sequence.

For example, expression of the spirdoin protein can be performed in E. coli by inserting the polynucleotide encoding a spirdoin polypeptide of the disclosure into plasmid vectors pFP202 and pFP204, which can be derived from the well-known vector pET11a. In these vectors, the dragline protein-coding gene is inserted in such a manner as to be operably linked to a promoter derived from bacteriophage T7. This promoter is joined with sequences derived from the lac operator of E. coli, which confers regulation by lactose or analogs (IPTG). The E. coli host strain BL21(DE3) contains a lambda prophage which carries a gene encoding bacteriophage T7 RNA polymerase. This gene is controlled by a promoter which is also regulated by lactose or analogs. In addition to the phage T7 promoter, the vectors pFP202 and pFP204 provide sequences which encode a C-terminal tail containing six consecutive histidine residues appended to the dragline protein-coding sequences. This tail provides a means of affinity purification of the protein under denaturing conditions through its adsorption to resins bearing immobilized Ni ions.

In addition, a sequence encoding an appropriate signal peptide (native or heterologous) can be incorporated into expression vectors. The choice of signal peptide or leader can depend on factors such as the type of host cells in which the recombinant polypeptide is to be produced. Examples of heterologous signal peptides that are functional in mammalian host cells include the signal sequence for interleukin (IL)-7 (see, U.S. Pat. No. 4,965,195); the signal sequence for IL-2 receptor (see, Cosman et al., Nature 312:768, 1984); the IL4 receptor signal peptide (see, EP 367,566); the type I IL-1 receptor signal peptide (see, U.S. Pat. No. 4,968,607); and the type II IL-1 receptor signal peptide (see, EP 460,846). A signal peptide that is functional in the intended host cells promotes extracellular secretion of the polypeptide. The signal peptide is cleaved from the polypeptide upon secretion of a polypeptide from the cell. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine or Lipofectamine-Plus lipid reagent (Gibco/BRL), can be used to transfect cells (Felgner et al., Proc. Natl. Acad. Sci. USA 84:7413, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487, 1990, describes several selection schemes, such as dihydrofolate reductase (DHFR) resistance. A suitable strain for DHFR selection can be CHO strain DX-B11, which is deficient in DHFR (Urlaub et al., Proc. Natl. Acad. Sci. USA 77:4216, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector are selected on the basis of resistance to these compounds.

Alternatively, gene products can be obtained via homologous recombination, or “gene targeting” techniques. Such techniques employ the introduction of exogenous transcription control elements (such as the CMV promoter or the like) in a particular predetermined site on the genome, to induce expression of an endogenous Spidroin of the disclosure. The location of integration into a host chromosome or genome can be easily determined by one of skill in the art, given the known location and sequence of the gene. In a preferred embodiment, the disclosure also contemplates the introduction of exogenous transcriptional control elements in conjunction with an amplifiable gene, to produce increased amounts of the gene product. The practice of homologous recombination or gene targeting is explained by Chappel in U.S. Pat. No. 5,272,071 (see also Schimke, et al. “Amplification of Genes in Somatic Mammalian cells,” Methods in Enzymology 151:85 (1987), and by Capecchi, et al., “The New Mouse Genetics: Altering the Genome by Gene Targeting,” TIG 5:70 (1989)).

Suitable host cells for expression of the polypeptide include eukaryotic, insect, plant and prokaryotic cells. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) (see, McMahan et al. EMBO J. 10: 2821, 1991), human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it may be possible to produce the polypeptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous polypeptides. Potentially suitable bacterial strains include, for example, Escherichia coli, Bacillus subtilis, Salmonella typhimurium, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain the functional polypeptide. Such covalent attachments may be accomplished using known chemical or enzymatic methods. The polypeptide may also be produced by operably linking a polynucleotide of the disclosure to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), as well as methods described in Summers and Smith, Texas Agricultural Experiment Station Bulletin No. 1555 (1987), and Luckow and Summers, Bio/Technology 6:47 (1988), incorporated herein by reference. Cell-free translation systems could also be employed to produce polypeptides using RNAs derived from nucleic acid constructs disclosed herein. A host cell that comprises an isolated polynucleotide of the disclosure, preferably operably linked to at least one expression control sequence, is a “recombinant host cell”.

In one embodiment, antagonists can be designed to reduce the level of endogenous Spidroin expression, e.g., using known antisense or ribozyme approaches to inhibit or prevent translation of Spidroin mRNA transcripts; triple helix approaches to inhibit transcription of Spidroin genes; or targeted homologous recombination to inactivate or “knock out” the Spidroin genes or their endogenous promoters or enhancer elements. Such antisense, ribozyme, and triple helix antagonists may be designed to reduce or inhibit either unimpaired or, if appropriate, mutant Spidroin activity. Such antagonists can be used as anti-insecticidals.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a mRNA having a Spidroin polynucleotide sequence. Absolute complementarity, although preferred, is not required. Oligonucleotides that are complementary to the 5′ end of the message, e.g., the 5′ untranslated sequence up to, and including, the AUG initiation codon, should work most efficiently at inhibiting translation. Antisense nucleic acids are preferably oligonucleotides ranging from 6 to about 50 nucleotides in length. The oligonucleotides can be DNA, RNA, chimeric mixtures, derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553, 1989; Lemaitre et al., Proc. Natl. Acad. Sci. 84:648, 1987; PCT Publication No. WO88/09810), or hybridization-triggered cleavage agents or intercalating agents (see, e.g., Zon, Pharm. Res. 5:539, 1988). The antisense molecules are delivered to cells, which express a transcript having a Spidroin polynucleotide sequence in vivo by, for example, direct injection into the tissue or cell derivation site, or modified antisense molecules, designed to target the desired cells (e.g., antisense linked to peptides or antibodies that specifically bind receptors or antigens expressed on the target cell surface) can be administered systemically. Preferred approach utilizes a recombinant DNA construct in which the antisense oligonucleotide is placed under the control of a strong pol III or pol II promoter.

Ribozyme molecules designed to catalytically cleave mRNA transcripts having a spidroin polynucleotide sequence prevent translation of Spidroin mRNA (see, e.g., PCT International Publication WO90/11364; U.S. Pat. No. 5,824,519). Ribozymes are RNA molecules possessing the ability to specifically cleave other single-stranded RNA. Because ribozymes are sequence-specific, only mRNAs with particular sequences are inactivated. There are two basic types of ribozymes namely, tetrahymena-type (Hasselhoff, Nature, 334:585, 1988) and “hammerhead”-type. Tetrahymena-type ribozymes recognize sequences, which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes. As in the antisense approach, ribozymes can be composed of modified oligonucleotides and delivered using a DNA construct “encoding” the ribozyme under the control of a strong constitutive pol III or pol II promoter.

Alternatively, endogenous Spidroin expression can be reduced by targeting DNA sequences complementary to a regulatory region of the target gene (e.g., the target gene promoter and/or enhancers) to form triple helical structures that prevent transcription of the target gene (see generally, Helene, Anticancer Drug Des., 6(6), 569, 1991; Helene, et al., Ann. N.Y. Acad. Sci., 660:27, 1992; and Maher, Bioassays 14(12), 807, 1992).

Antisense, ribozyme, and triple helix molecules of the disclosure may be prepared by any method known in the art for the synthesis of DNA and RNA molecules and include techniques for chemically synthesizing oligodeoxyribonucleotides and oligoribonucleotides such as, for example, solid phase phosphoramidite chemical synthesis using an automated DNA synthesizer available from Biosearch, Applied Biosystems. Phosphorothioate oligonucleotides may be synthesized by the method of Stein et al., Nucl. Acids Res. 16:3209, 1988. Methylphosphonate oligonucleotides can be prepared by use of controlled pore glass polymer supports (Sarin et al., Proc. Natl. Acad. Sci. U.S.A. 85:7448, 1988). Alternatively, RNA molecules may be generated by in vitro and in vivo transcription of DNA sequences encoding the antisense RNA molecule.

As used herein, a “transgenic organism” is a non-human organism that includes a transgene that is inserted into an embryonal cell and becomes a part of the genome of the organism that develops from that cell, or an offspring of such an organism. Any non-human organism that can be produced by transgenic technology is included in the disclosure. Typical organisms can include non-human animals, silk worms and other insects, and plant cells into which a Spidroin transgene has been inserted.

A “transgene” is a polynucleotide that comprises one or more selected sequences (e.g., encoding a Spidroin, encoding ribozymes that cleave Spidroin mRNA, encoding an antisense molecule to a Spidroin mRNA, encoding a mutant Spidroin sequence, and the like) to be expressed in a transgenic organism. The polynucleotide is partly or entirely heterologous, i.e., foreign, to the transgenic animal, plant or insect, or homologous to an endogenous gene of the transgenic animal, plant or insect, but which is designed to be inserted into the genome at a location which differs from that of the natural gene. A transgene may include one or more promoters and any other DNA sequences, such as introns, necessary for expression of the selected DNA, all operably linked to the selected DNA, and may include an enhancer sequence.

The transgenic organism can be used for the production of spider silk dragline commprising a Spidroin polypeptide or fragment thereof. For example, a transgenic organism can be used for large scale production of silk materials using the polynucleotides of the disclosure. Such silk materials can be harvested and used for the generation of textiles, biomaterials and the like. In another aspect, the transgenic organism can be used in order to identify the impact of increased or decreased Spidroin levels on a particular pathway or phenotype. Protocols useful in producing such transgenic animals are known in the art (see, e.g., Brinster, et al., Proc. Natl. Acad. Sci. USA 82:4438, 1985; Jaenisch, Proc. Natl. Acad. Sci. USA 73:1260, 1976; Hogan, et al., 1986, Manipulating the Mouse Embryo, Cold Spring Harbor Laboratory Press, Cold Spring Harbor, N.Y.; Jahner, et al., Proc. Natl. Acad. Sci. USA 82:6927, 1985; Van der Putten, et al., Proc Natl. Acad. Sci. USA 82:6148; Steward, et al., EMBO J., 6:383, 1987; Jahner, et al., Nature, 298:623, 1982).

In another embodiment, antibodies that are immunoreactive with the polypeptides of the disclosure are provided herein. The Spidroin polypeptides, fragments, variants, fusion polypeptides, and the like, as set forth above, can be employed as “immunogens” in producing antibodies immunoreactive therewith. Such antibodies specifically bind to the polypeptides via the antigen-binding sites of the antibody. Specifically binding antibodies are those that will specifically recognize and bind with Spidroin family polypeptides, homologues, and variants, but not with other molecules. In one embodiment, the antibodies are specific for polypeptides having a spidroin amino acid sequence of the disclosure and do not cross-react with other polypeptides.

More specifically, the polypeptides, fragment, variants, fusion polypeptides, and the like contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding. Epitopes can be identified by any of the methods known in the art. Additionally, epitopes from the polypeptides of the disclosure can be used as research reagents, in assays, and to purify specific binding antibodies from substances such as polyclonal sera or supernatants from cultured hybridomas. Such epitopes or variants thereof can be produced using techniques known in the art such as solid-phase synthesis, chemical or enzymatic cleavage of a polypeptide, or using recombinant DNA technology.

Both polyclonal and monoclonal antibodies to the polypeptides of the disclosure can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kosbor et al., Immunology Today 4:72, 1983; Cole et al., Proc. Natl. Acad. Sci. USA 80:2026, 1983); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the disclosure are also contemplated herein. Such hybridomas can be produced and identified by conventional techniques. For the production of antibodies, various host animals may be immunized by injection with a Spidroin and Spidroin polypeptide, fragment, variant, or mutants thereof. Such host animals may include, but are not limited to, rabbits, mice, and rats, to name a few. Various adjutants may be used to increase the immunological response. Depending on the host species, such adjutants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies may be of any immunoglobulin class including IgG, IgM, IgE, IgA, IgD, and any subclass thereof.

Antibody fragments, which recognize specific epitopes, may be generated by known techniques. For example, such fragments include but are not limited to: the F(ab′)₂ fragments which can be produced by pepsin digestion of the antibody molecule and the Fab fragments which can be generated by reducing the disulfide bridges of the (ab′)₂ fragments. Alternatively, Fab expression libraries may be constructed (Huse et al., Science, 246:1275, 1989) to allow rapid and easy identification of monoclonal Fab fragments with the desired specificity. Techniques described for the production of single chain antibodies (U.S. Pat. No. 4,946,778; Bird, Science 242:423, 1988; Huston et al., Proc. Natl. Acad. Sci. USA 85:5879, 1988; and Ward et al., Nature 334:544, 1989) can also be adapted to produce single chain antibodies against polypeptides containing Spidroin amino acid sequences.

The antibodies of the disclosure can also be used in assays to detect the presence of the polypeptides or fragments of the disclosure, either in vitro or in vivo. The antibodies also can be employed in purifying polypeptides or fragments of the disclosure by immunoaffinity chromatography.

The disclosure provides methods for identifying agents that modulate Spidroin activity or expression. Such methods included contacting a sample containing a spidroin polypeptide or polynucleotide with a test agent under conditions that allow for the test agent and the polypeptide or polynucleotide to interact and measuring the expression or activity of a Spidroin polypeptide in the presence or absence of the test agent.

In one embodiment, a cell containing a Spidroin polynucleotide is contacted with a test agent under conditions such that the cell and test agent are allowed to interact. Such conditions typically include normal cell culture conditions consistent with the particular cell type being utilized and which are known in the art. It may be desirable to allow the test agent and cell to interact under conditions associated with increased temperature or in the presence of regents that facilitate the uptake of the test agent by the cell. A control is treated similarly but in the absence of the test agent. Alternatively, the Spidroin activity or expression may be measured prior to contact with the test agent (e.g., the standard or control measurement) and then again following contact with the test agent. The treated cell is then compared to the control and a difference in the expression or activity of Spidroin compared to the control is indicative of an agent that modulates Spidroin activity or expression.

When Spidroin expression is being measured, detecting the amount of mRNA encoding a spidroin polypeptide in the cell can be quantified by, for example, RT-PCR or Northern blot. Where a change in the amount of Spidroin polypeptide in the sample is being measured, detecting Spidroin by use of anti-Spidroin antibodies can be used to quantify the amount of Spidroin polypeptide in the cell using known techniques.

A test agent can be any molecule typically used in the modulation of protein activity or expression and includes, for example, small molecules, chemicals, peptidomimetics, antibodies, peptides, polynucleotides (e.g., antisense or ribozyme molecules), and the like. Accordingly, agents developed by computer based design can be tested in the laboratory using the assay and methods described herein to determine the activity of the agent on the modulation of Spidroin activity or expression. Modulation of Spidroin includes an increase or decrease in activity or expression or strength of the resulting fibrous material.

Uses of Spidroin polypeptides and peptide fragments thereof include, but are not limited to, the following: delivery agents; textile materials; biomaterials for wound repair; biomaterials for tissue engineering; puncture resistant materials; molecular weight and isoelectric focusing markers; and preparation of antibodies.

The spider silk compositions provided herein find uses in the textile industry (e.g., as filaments, yarns, ropes, and woven material). Such materials made using the methods and compositions described herein will take advantage of the extreme toughness, tensile strength, and extensibility of silk. In addition, the polypeptides of the disclosure can be used in pliant energy absorbing devices including armor and bumpers. Besides the mechanical properties of spider silk, silk is proteinaceous (thus not petroleum-based like nylon or Kevlar). Accordingly, the polypeptides of the disclosure provide biocompatible and biodegradable material useful in various industries including textiles and medicine. For example, the supercontraction ability of dragline silk can be beneficial for sutures that can tighten, compression bandages, or space minimizing packaging. Additionally the polypeptides can be used in the generation of scaffolds and material in tissue engineering, implants and other cell scaffold-based materials. The polypeptides of the disclosure can be used in the generation of biomaterials comprising other proteinacious substances (e.g., as a collagen and silk material combination).

For compositions of the disclosure which are useful for tissue repair or regeneration, the therapeutic method includes administering or contacting a site in need of wound repair with a biomaterial comprising a Spidroin polypeptide or fragment, physiologically acceptable form of the composition can be used topically, systematically, locally or in association with an implant or device.

Further encompassed by the disclosure are systems and methods for analyzing Spidroin polypeptides comprising identifying and/or characterizing one or more Spidroin polypeptides, encoding nucleic acids, and corresponding genes, these systems and methods comprise a data set representing a set of one or more Spidroin molecules, or the use thereof. Accordingly, the disclosure provides a computer readable medium having stored thereon a member selected from the group consisting of a polynucleotide comprising a sequence as set forth in SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106; a polypeptide comprising a sequence as set forth in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107; a set of polynucleotide sequences wherein at least one of said sequences comprises a sequence as set forth in SEQ ID Nos: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106; and a set of polypeptide sequences wherein at least one of said sequences comprises a sequence as set forth in SEQ ID Nos: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 or 107.

One embodiment of the disclosure comprises a computing environment and a plurality of algorithms selectively executed to analyze a polypeptide or polynucleotide of the disclosure. Examples of analyses of a spidroin polypeptide include, without limitation, displaying the amino acid sequence of a polypeptide in the set, comparing the amino acid sequence of one polypeptide in the set to the amino acid sequence of another polypeptide in the set, predicting the structure of a polypeptide in the set, determining the nucleotide sequences of nucleic acids encoding a polypeptide in the set, and identifying a gene corresponding to a polypeptide in the set.

cDNA transcripts, partial-length genes, and full-length genes were deduced from multiple species of spiders. These nucleotide sequences encode spider silk proteins that can be used directly (expressed with no modification) or with modifications (such as manipulating codons and adding promoters and tags to facilitate high yield and purification) for the production of biomaterials with novel combinations of properties. Some of the sequences described below include previously unknown gene regions for partially described spidroins.

### EXAMPLES

### Taxonomic Sampling

Our taxonomic sampling was aimed at covering phylogenetic diversity and surveying a variety of web architectures. The mesothele representative, Liphistius malayanus, constructs a subterranean burrow with a trapdoor and radiating sensory lines. Six species of mygalomorphs were sampled. From the Atypoidea Glade, which is the sister group to remaining mygalomorphs (Hedin and Bond, 2006; Ayoub et al., 2007a), the following species were selected, with web constructs in parentheses: Megahexura fulva (sheet-web), Hexura picea (sheet-web), Sphodros rufipes (purse-web), and Antrodiaetus riversi (burrow with turret-like entrance). Two non-atypoid mygalomorphs were sampled from the family Theraphosidae, Aphonopelma seemanni, a ground dweller (burrow/sheet web), and Poecilotheria regalis, an arboreal species (sheet web). The lamp-shade web spider, Hypochilus thorelli, which is a member of the basal araneomorph lineage, Paleocribellatae was also included.

cDNA Library Construction and Screening.

The cDNA library construction methods described in Garb et al. (2007) were used. Briefly, each spider was anesthetized with CO₂ and then the entire set of silk glands was removed intact. The silk glands were frozen in liquid nitrogen and stored at −80° C. With the exception of the two theraphosids, glands from multiple individuals of the same species were combined to obtain sufficient tissue. Total RNA was extracted using TRIzol (Invitrogen, Carlsbad, Calif.) and the RNeasy Minikit (Qiagen, Valencia, Calif.). mRNA was isolated from total RNA using Dynal magnetic beads with oligo-(dT) anchors (Invitrogen). Double stranded cDNA was constructed using the Superscript Choice protocol (Invitrogen), and then size selected for large fragments using Chroma Spin 1000 columns (Clontech, Mountain View, Calif.). The size-selected cDNA was ligated into pZErO 2.0 vectors that had been digested with EcoRV, and then transformed into TOP10 Escherichia coli (Invitrogen). For each species, ˜1400-1700 cDNA clones were arrayed into 96-well microtiter plates. The libraries were stored at −80° C.

Approximately one third of each library was screened using the method of Beuken, Vink, and Bruggeman (1998) and sequenced clones containing inserts 500 base pairs with T7 and Sp6 universal primers. Sequences were compared to the NCBI nr database using BLASTX (Altschul et al., 1990) to identify potential silk homologs. Libraries were also replicated onto nylon filters and probed with γ³² P-labeled oligonucleotides. All libraries were screened with GCDGCDGCDGCDGCDGC (SEQ ID NO:109) and CCWGCWCCWGCWCCWGCWCC (SEQ DI NO:110), which were designed based on motifs common to spidroins (Gatesy et al., 2001; Garb and Hayashi, 2005; Garb et al., 2006). Additionally, libraries were screened with taxon specific probes designed from the end sequences of the size-selected clones. For putative Liphistius Egg Case Proteins (ECPs), the following probes were developed: 1) TAGTAATAAGTTCCATCGCA (SEQ ID NO:111), 2) GCAAGGATTATAAGGATG (SEQ ID NO:112), 3) CTTACCCTCTCCACATTCAGT (SEQ ID NO:113), 4) GGTTTAACTTTGTTGGCGTC (SEQ ID NO:114), 5) GGGGTCGTAAAATGATTGATA (SEQ ID NO:115), 6) ACATTGGTTCTTTTTGTAGCA (SEQ ID NO:116), and 7) GTTCTTGTCGTAGCATTTGTA (SEQ ID NO:117). Probes designed from putative spidroins were 8) AAAAGCAGTGGCAGTGGCTTC (SEQ ID NO:118), 9) CCCCTAAAATAGGTATTCTGATA (SEQ ID NO:119) (8, 9 for Liphistius); 10) GCCGTATGATGCTGACTGTAG (SEQ ID NO:120), 11) TGCTGATGCGGCGGCTTG (SEQ ID NO:121), 12) GCTTGCATAGGCTGAGGC (SEQ ID NO:122) (10-12 for Megahexura); 13) TATATCAGTTCCATATGGTCC (SEQ ID NO:123), 14) GGATCGAAAACGTTGTGAAA (SEQ ID NO:124), 15) AGCTGCTTCATTTGCTGTGTT (SEQ ID NO:125), 16) CTTACCACAGGCGTAACC (SEQ ID NO:126) (13-16 for Hexura); 17) GCCGCTGCATCGGCGTAGGC (SEQ ID NO:127), 18) AATGCAAATGCGATGGCATA (SEQ ID NO:128), 19) CAACACACCACTCAATCCAGA (SEQ ID NO:129) (17-19 for Sphodros); 20) GCTCCTTCWCTMCCATATCCTCC (SEQ ID NO:130), 21) GCTTCAGCATAYGCTTTTGC (SEQ ID NO:131), 22) TCTRGCATAACTAGCGGCATC (SEQ ID NO:132), 23) GTAAACTGATTCGAATTCGTC (SEQ ID NO:133) (20-23 for Antrodiaetus); 24) TTATCACACATCATTTTTCC (SEQ ID NO:134) (24 for Aphonopelma); 25) CATGGCAGAGGGTATCAGGT (SEQ ID NO:135), 26) AGTGTAATTTGCAATGCC (SEQ ID NO:136), 27) GCAAGAGCAATGGCGTTTCC (SEQ ID NO:137), 28) ATAGGCATAAGCACCAGCGTT (SEQ ID NO:138), 29) GTAAGCATAAGCCTCGGCTCC (SEQ ID NO:139) (25-29 for Poecilotheria); 30) AGCTCCWGCACTTGCNCCACT (SEQ ID NO:140) (30 for Hypochilus).

All positive clones were sequenced using T7 and Sp6 universal primers. Based on these sequences, clones that had the same translated carboxyl (C) terminal region were grouped with each other. For each group, the clone with the longest insert was selected for complete characterization. Because the inserts contained repetitive nucleotide sequence, a primer walking approach was not feasible. Instead, each selected clone was bidirectionally sequenced in its entirety using the transposon-based GPS-1 Genome Priming System (NEB, Ipswich, Mass.) or EZ-Tn5 Kit (Epicentre, Madison, Wis.).

Alignment of Egg Case Proteins. Putative Liphistius malayanus ECPs were aligned with Latrodectus hesperus ECP-1 (AY994149) and ECP-2 (DQ341220) using MUSCLE with default settings (Edgar, 2004). The alignment was imported into GeneDoc 2.7.0 (Nicholas and Nicholas, 1997) and physiochemically conserved sites were highlighted.

Phylogenetic Analyses.

Phylogenetic analyses were conducted on a dataset of C-terminal encoding regions from published spidroins and those reported here. Spidroins from GenBank were selected to represent different silk glands and phylogenetic diversity. From Araneomorphae, Argiope trifasciata AcSp1 (accession number AY426339), Flag (AF350264), and pyriform (GQ980328; referred to as PySp1 in this paper); Deinopis spinosa Flag (DQ399325), fibroin 1a (DQ399326), fibroin 1b (DQ399327), fibroin 2 (DQ399323), MaSp2a (DQ399328), MaSp2b (DQ399329), MiSp1 (DQ399324), and TuSp1 (AY953073); Diguetia canities MaSp-like (HM752567) and MaSplike2 (HM752565); Dolomedes tenebrosus Dtfib1 (AF350269) and Dtfib2 (AF350270); Latrodectus hesperus AcSp1 (EUO25854), MaSp1 (DQ409057), MaSp2 (EF595245), PySp1 (FJ973621), and TuSp1 (AY953070); Nephila clavipes Flag (AF027973), MaSp1 (AY654292), MaSp2 (M92913), MiSp1 (AF027735), pyriform (GQ980330; referred to here as PySp1), and TuSp1 (AY855102); Peucetia viridans MaSp1 (GU306168); Plectreurys tristis fibroin 1 (AF350281), fibroin 2 (AF350282), fibroin 3 (AF350283), and fibroin 4 (AF350284); and Uloborus diversus AcSp1 (DQ399333), MaSp1 (DQ399331), MaSp2 (DQ399334), MiSp (DQ399332), and TuSp1 (AY953072). From Mygalomorphae, we included Aliatypus gulosus (originally identified as A. plutonis) fibroin 1 (EU117159); Aptostichus sp. fibroin 1 (EU117160) and fibroin 2 (EU117161); Avicularia juruensis spidroin 1a (EU652181; referred to as fib1a in this study), 1b (EU652182; referred to as fib1b in this study), and 1c (EU652183; referred to as fib1c in this study); Bothriocyrtum californicum fibroin 1 (EU117162), fibroin 2 (EU117163), and fibroin 3 (EU117164); and Euagrus chisoseus fibroin 1 (AF350271). The C-terminal regions were aligned using MUSCLE under default parameters (Edgar, 2004) followed by manual adjustment. C-terminal encoding DNA sequences were aligned according to the amino acid alignment with PAL2NAL (Suyama, Torrents, and Bork, 2006) were included.

Avicularia juruensis spidroin 2 (EU652184) was not included in the final analyses. A Blastn search of the Avicularia spidroin 2 C-terminal region resulted in only two hits. These hits (accessions AF350267, AY365020) were MaSp2 sequences from two species of the orbicularian, Argiope, and had extremely strong E values (<1e-63). Phylogenetic analysis grouped this sequence with araneoid major ampullate sequences (Bittencourt et al., 2010). This result could not be corroborated as close relatives of Avicularia spidroin 2 were not found in any of 10 mygalomorph cDNA libraries (Gatesy et al., 2001; Garb et al., 2007; this study); nor did Prosdocimi et al. (2011) recover major ampullate-like spidroins from the silk gland transcriptome of the mygalomorph, Actinopus sp.

A phylogenetic analyses was conducted using maximum likelihood (ML) and Bayesian methods. Analyses were conducted on DNA data with gaps coded using the ‘Simple’ method following Simmons and Ochoterena (2000). Analyses were conducted through the CIPRES web server (Miller, Pfeiffer, and Schwartz, 2010). Likelihood searches for the best tree and bootstrap were performed simultaneously with 1000 replicates using RAxML v. 7.2.8 (Stamatakis, 2006 a,b; Stamatakis, Hoover, and Rougemont, 2008). Analyses were performed with the data partitioned by codon position, using the GTR+γ model for each partition, following RAxML program author recommendations. Coded gaps were treated as binary data and as a separate data partition.

Bayesian analyses were conducted using MrBayes v. 3.1.2 (Huelsenbeck and Ronquist, 2001; Ronquist and Huelsenbeck, 2003). DNA substitution models were determined for each codon position (position 1: HKY+I+γ, position 2: GTR+I+γ, position 3: GTR+γ) using MrModeltest v. 2.3 (Nylander, 2004). The restriction site (binary) model with variable ascertainment bias was used for the coded gap characters (Ronquist, Huelsenbeck, and van der Mark, 2005). Two simultaneous searches were run for at least 10 million generations, with trees and parameters sampled from four MCMC chains every 1000^{th} generation. Partitions (codon positions and binary characters) were unlinked and substitution rates of evolution among partitions were allowed to vary. Analyses were considered complete when the standard deviation of split frequencies between the two searches was below 0.01 (Ronquist, Huelensenbeck, and van der Mark, 2005). The first forty percent of samples were treated as burnin and discarded. Bayesian posterior probabilities (PP) were used to assess clade support.

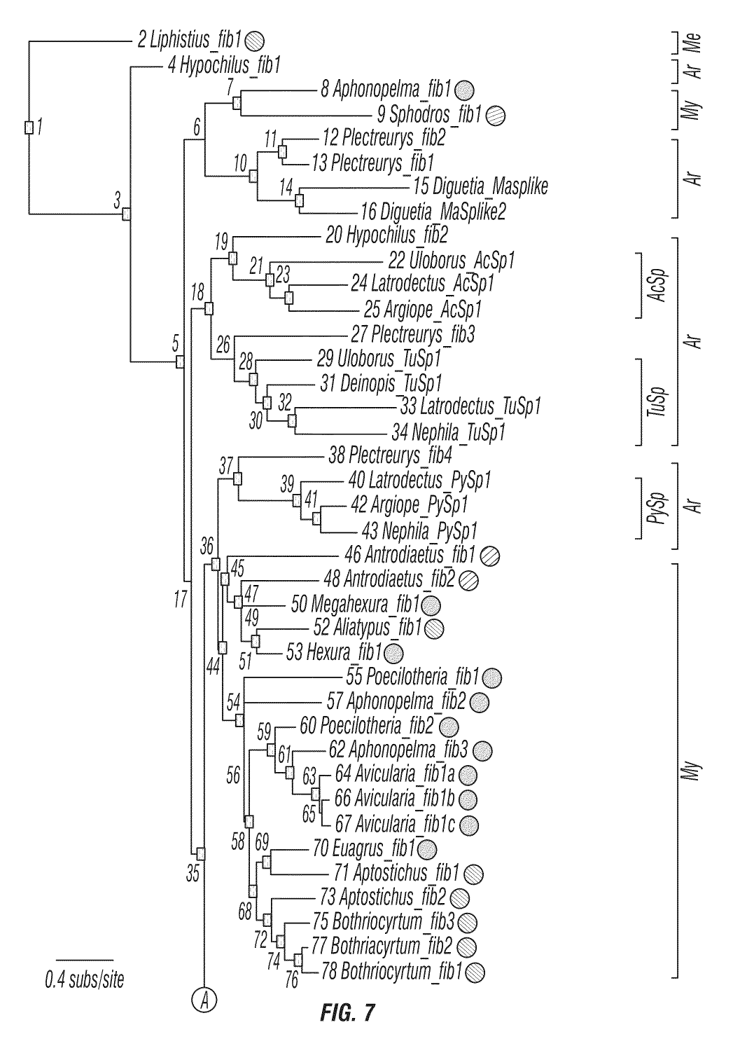

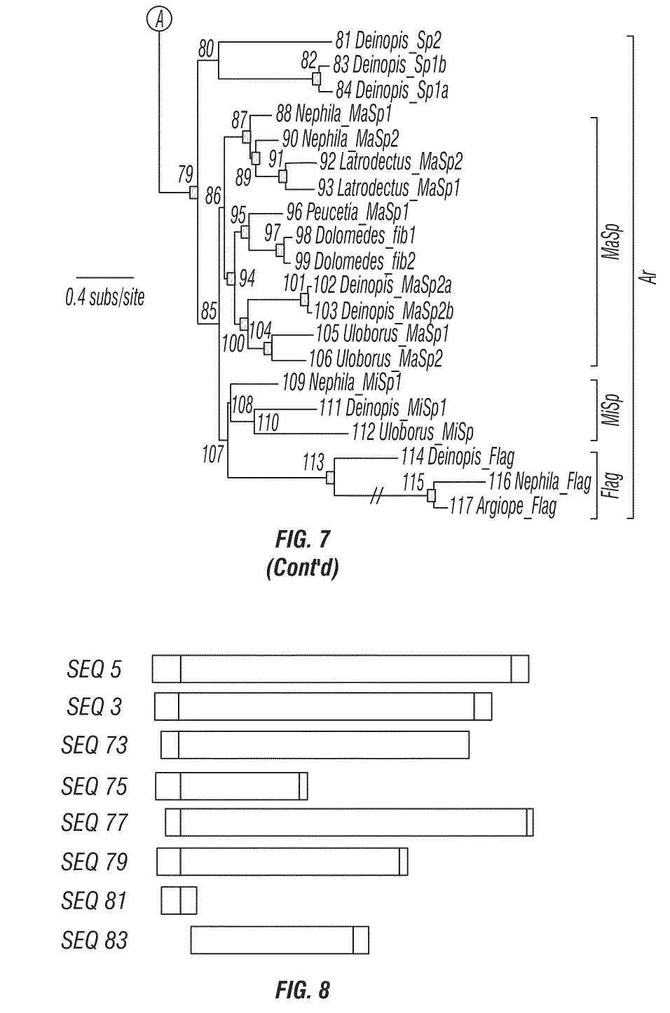

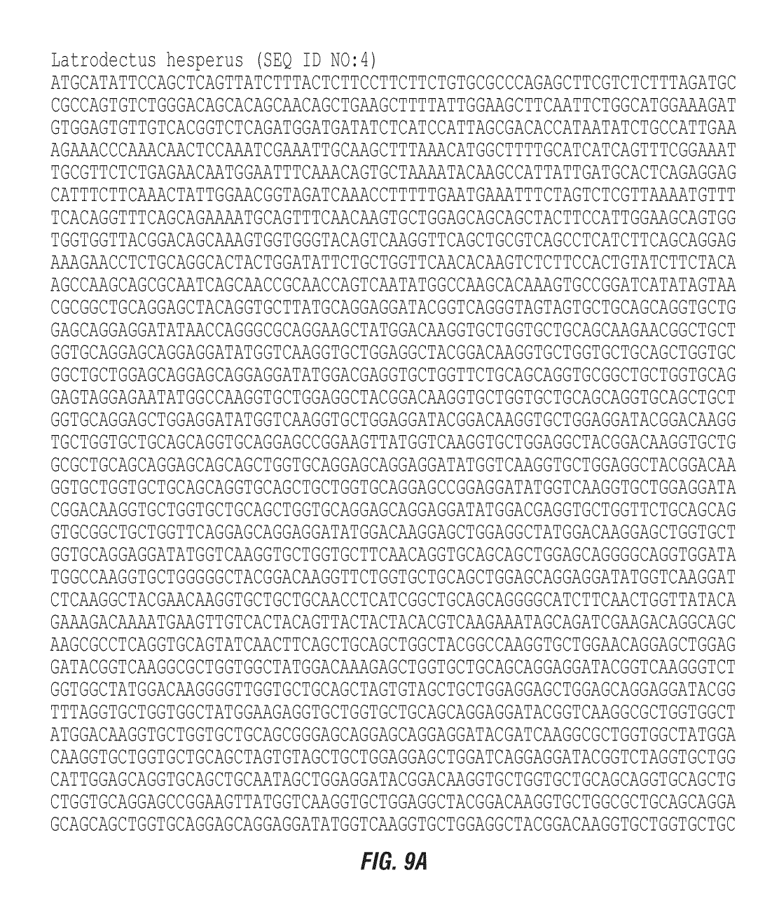

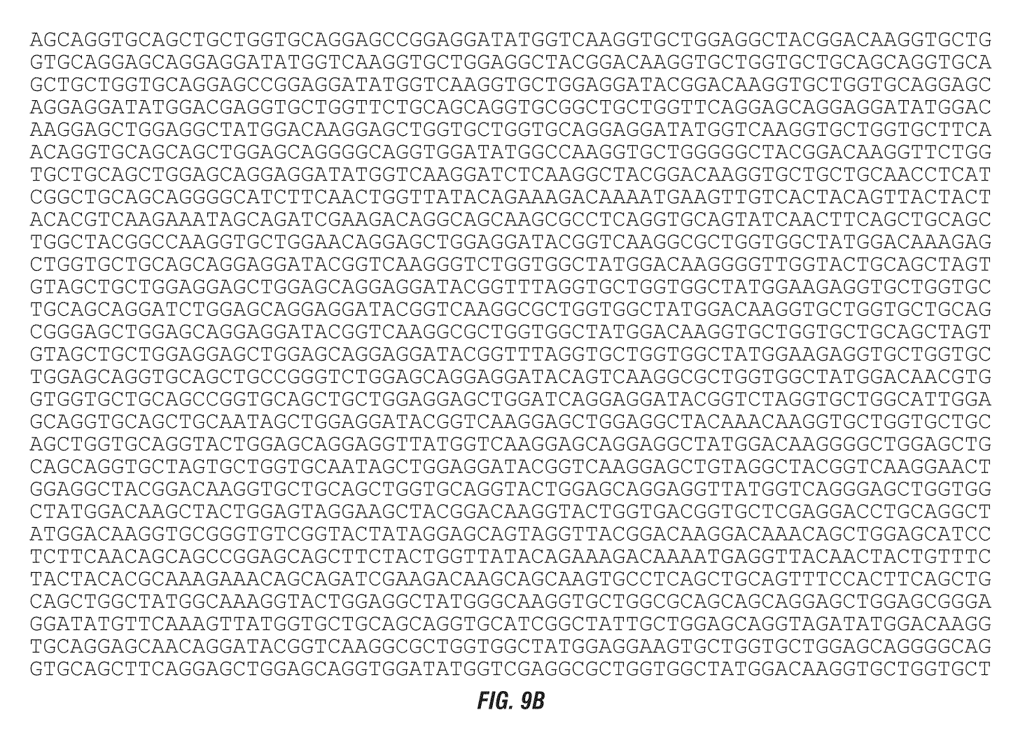

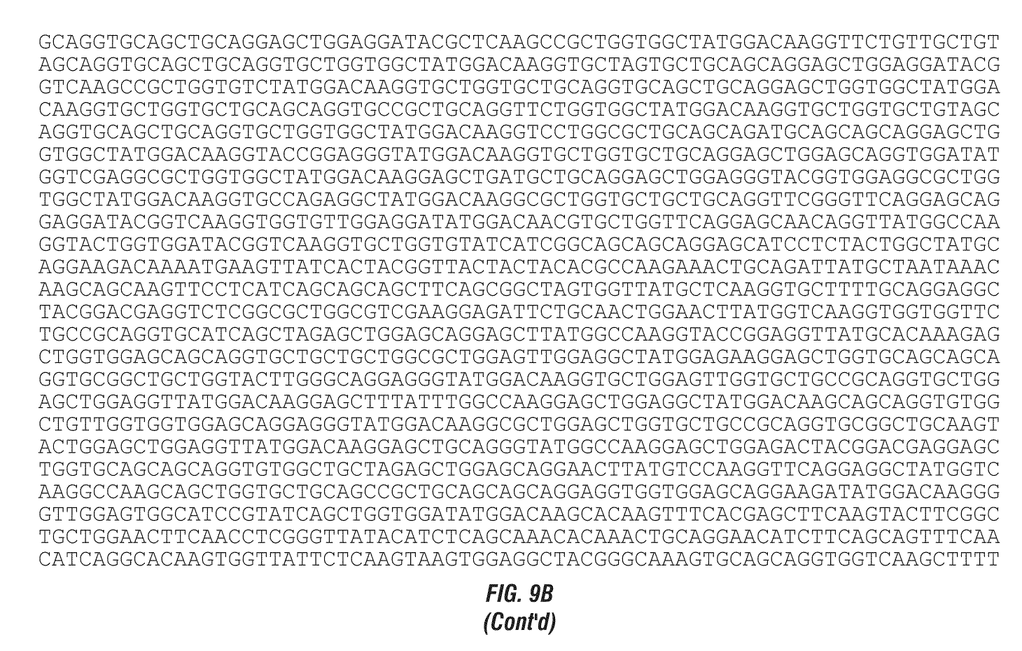

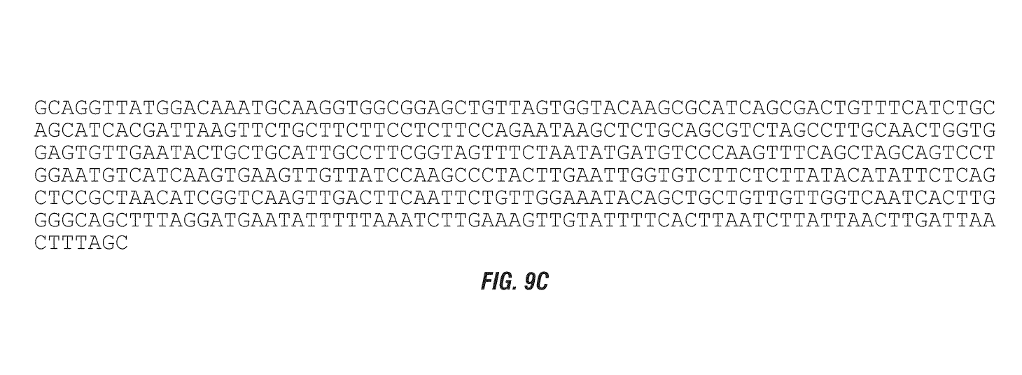

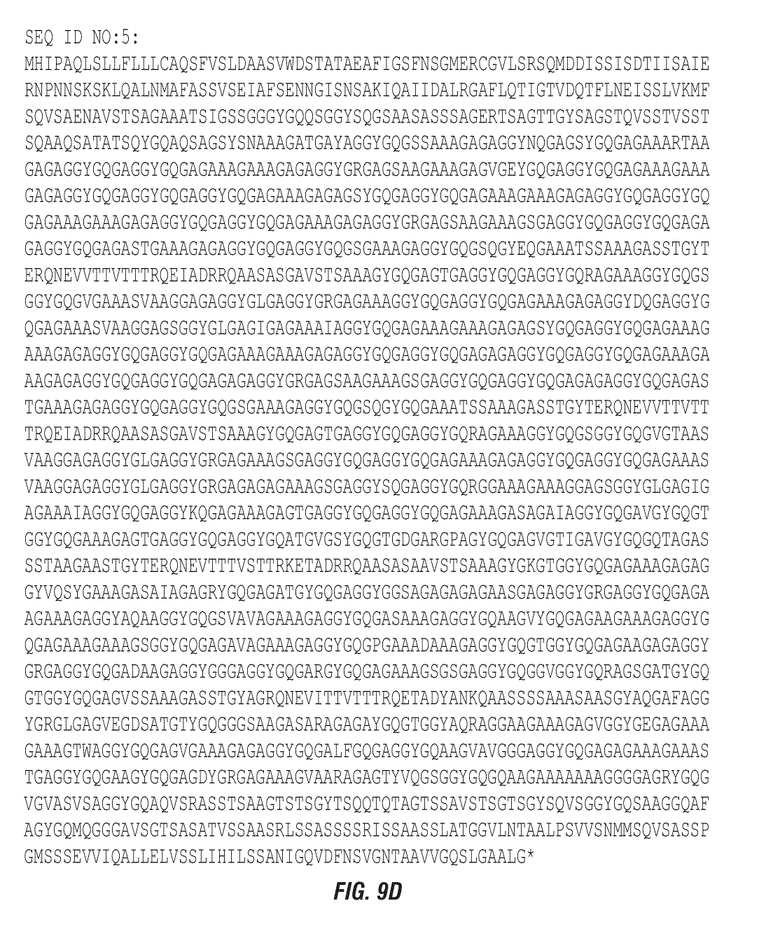

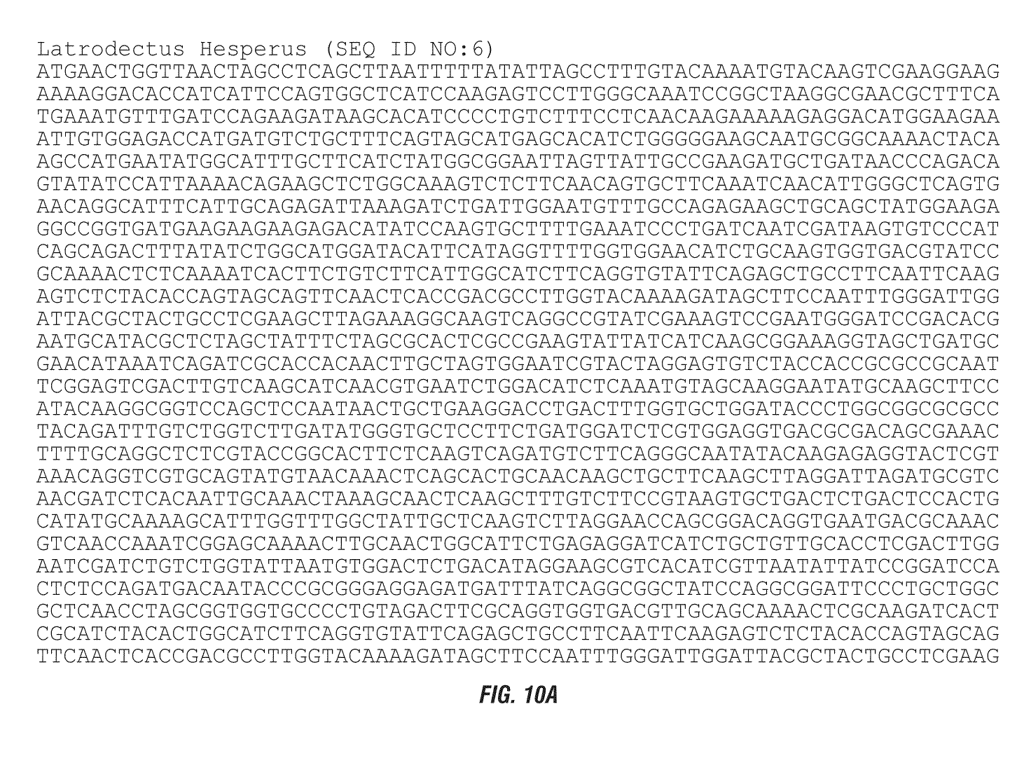

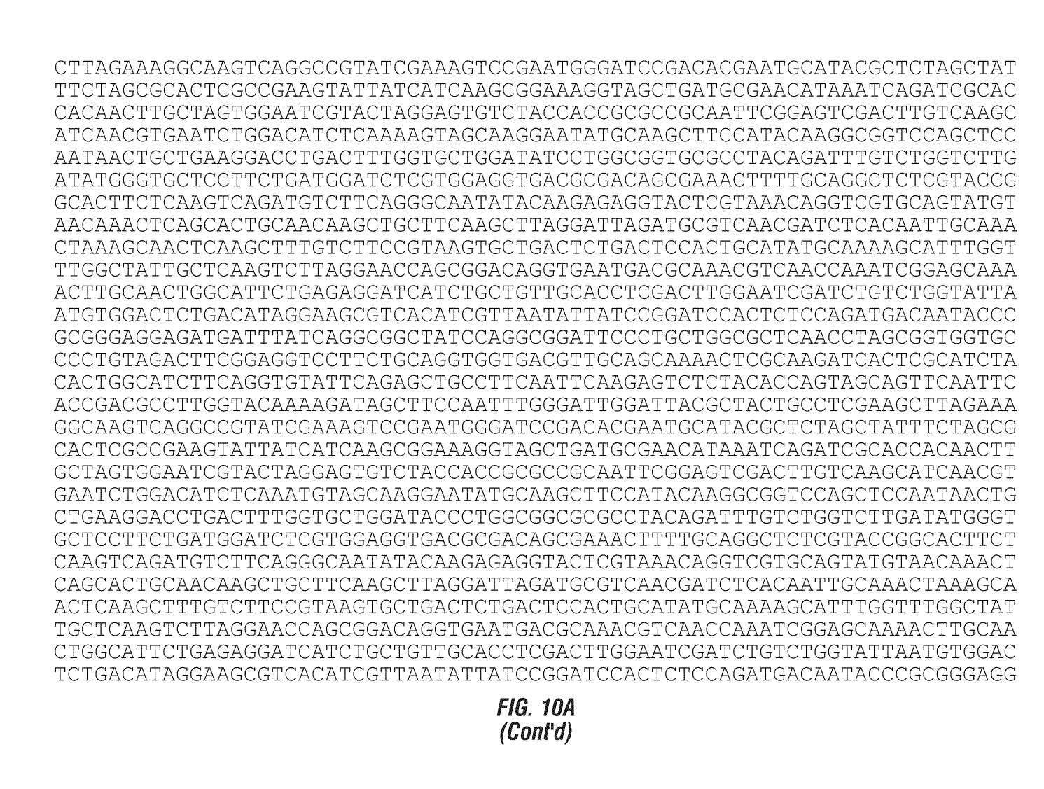

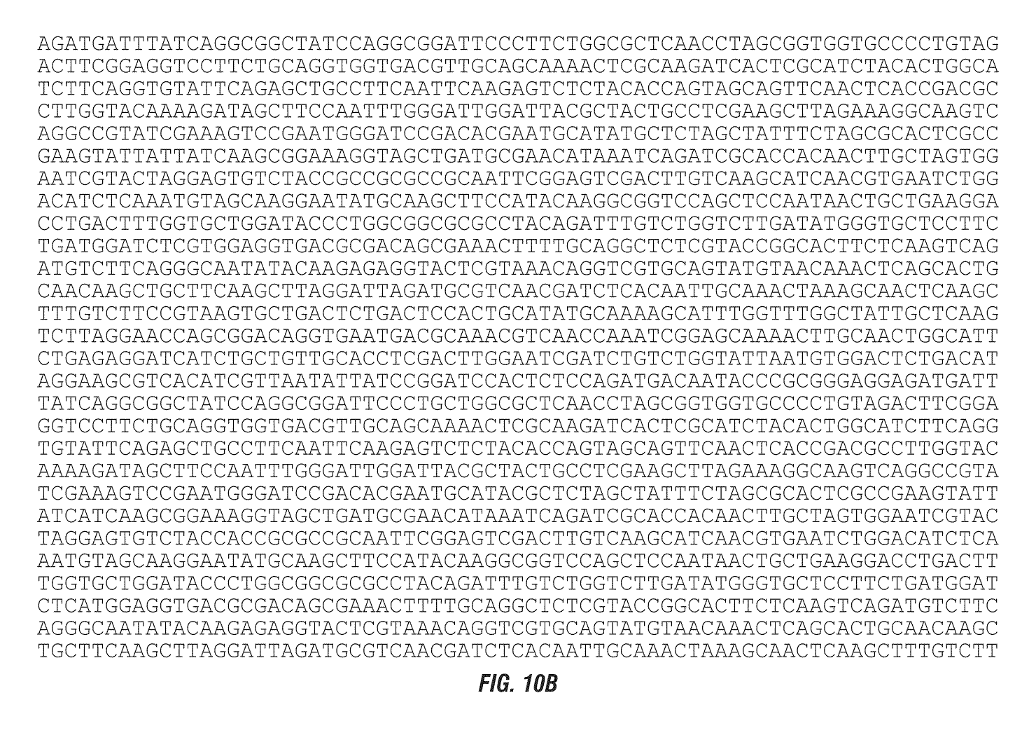

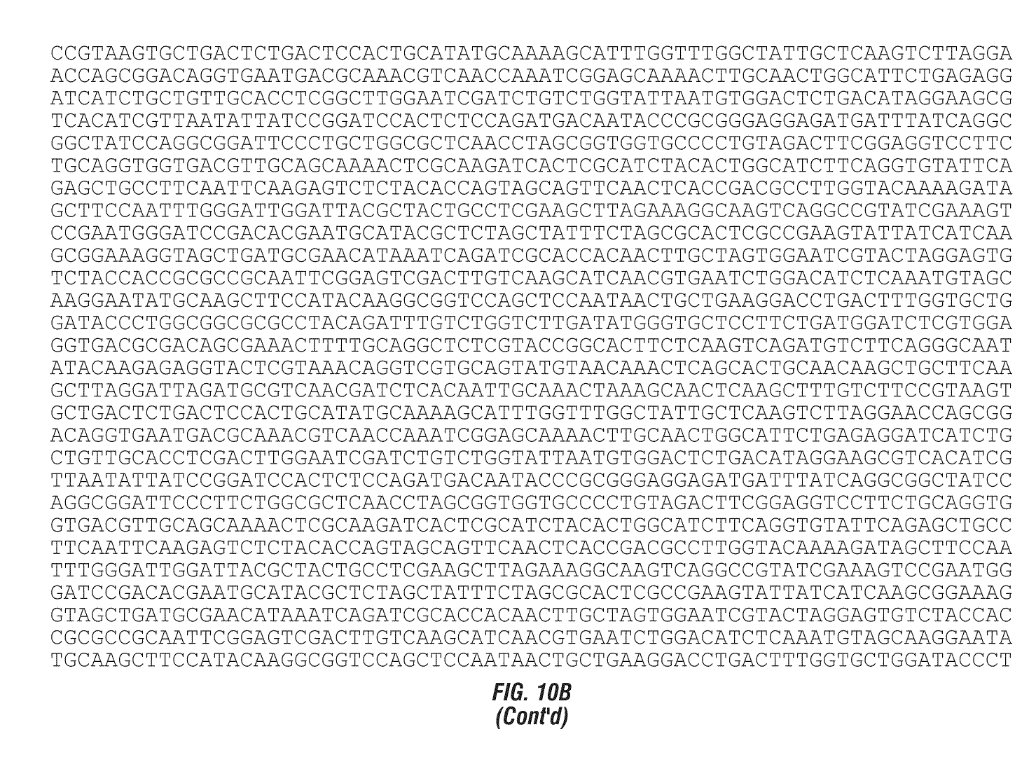

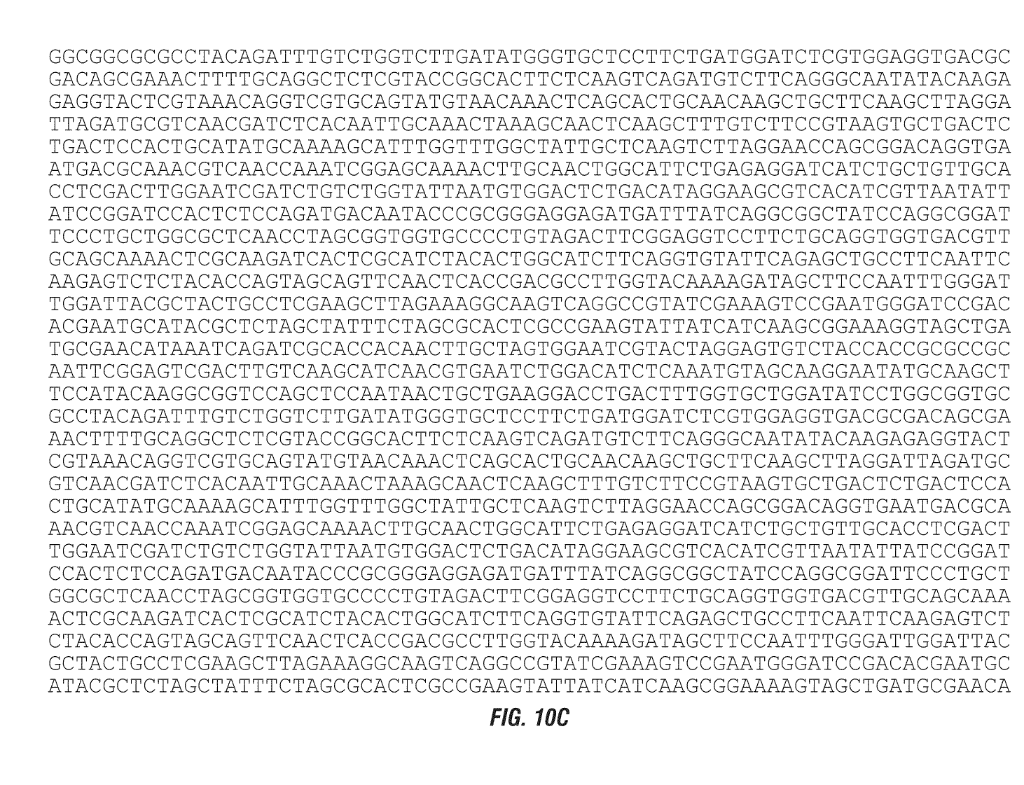

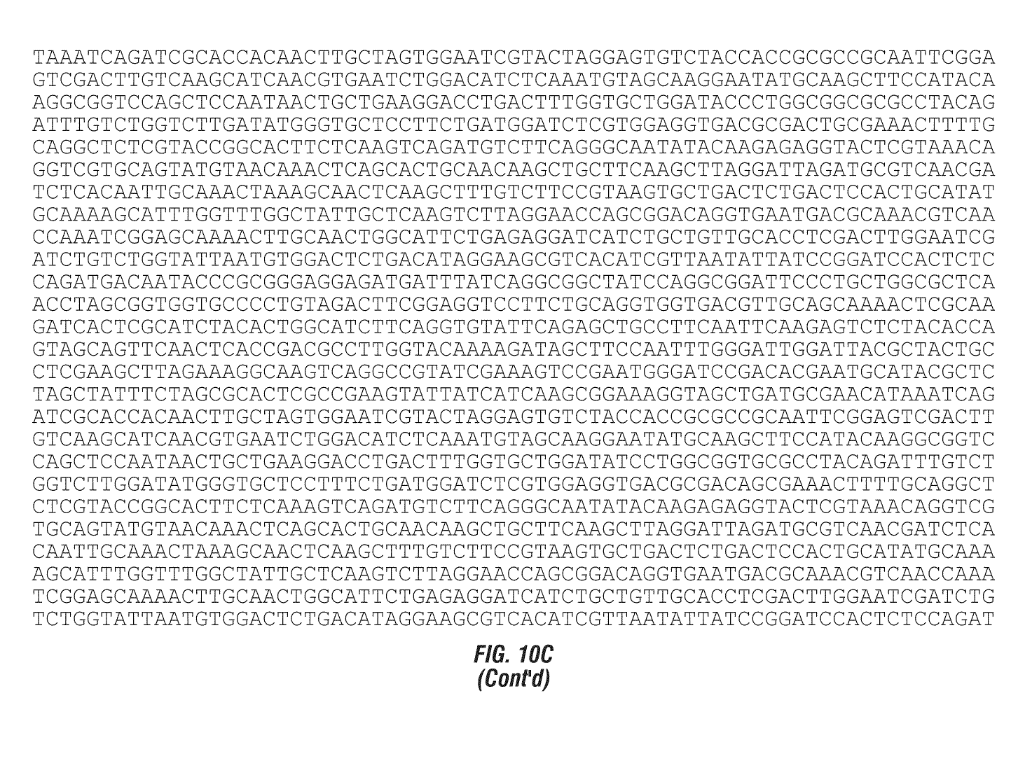

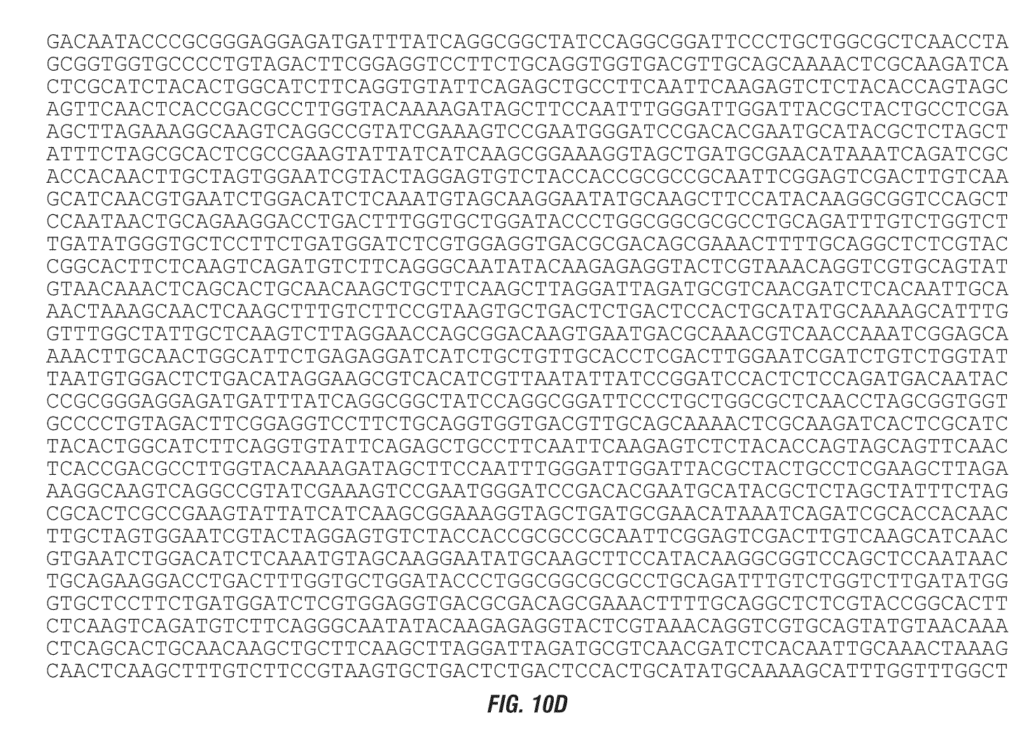

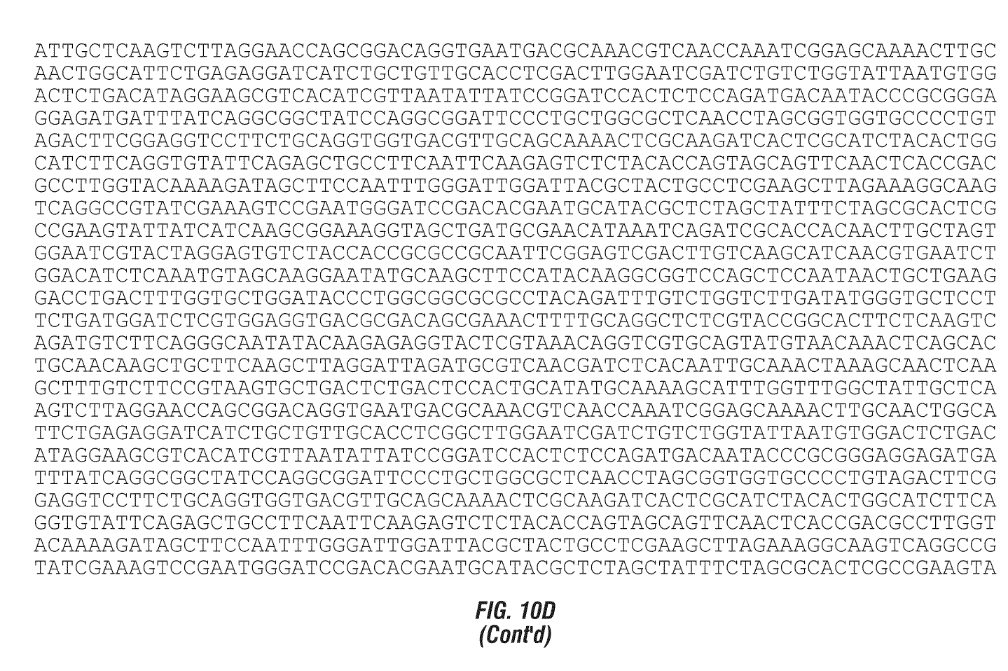

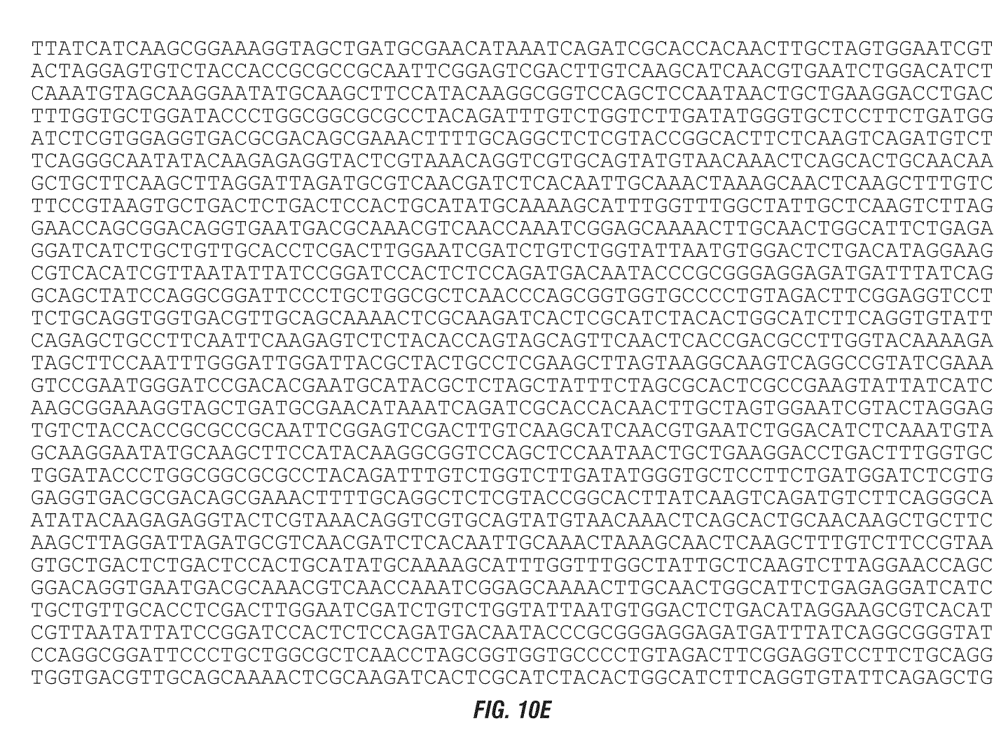

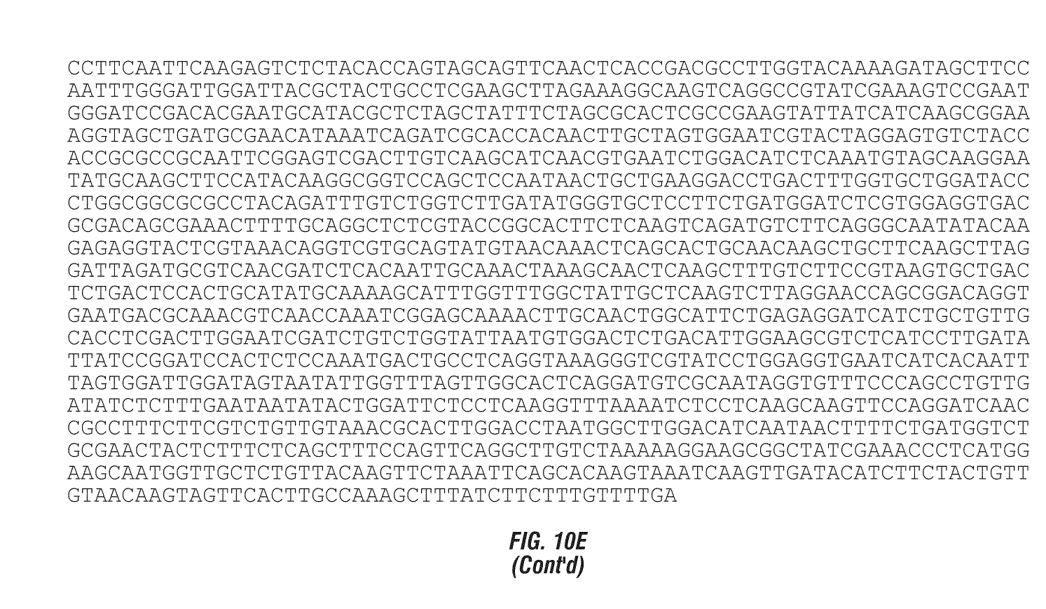

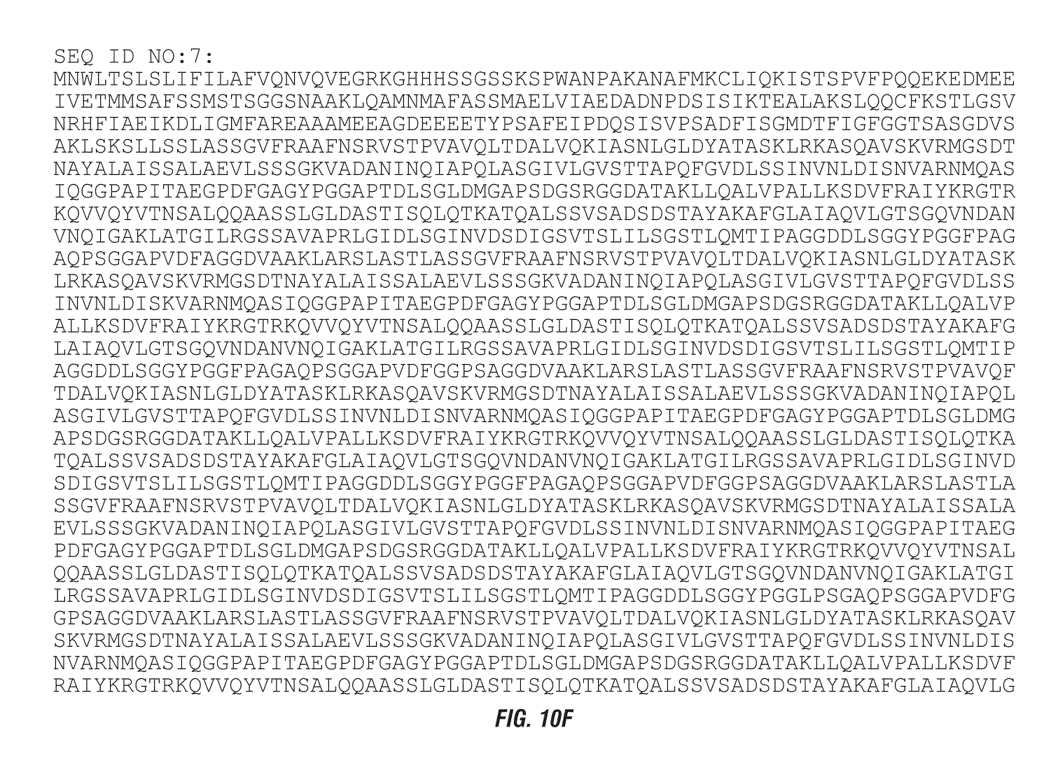

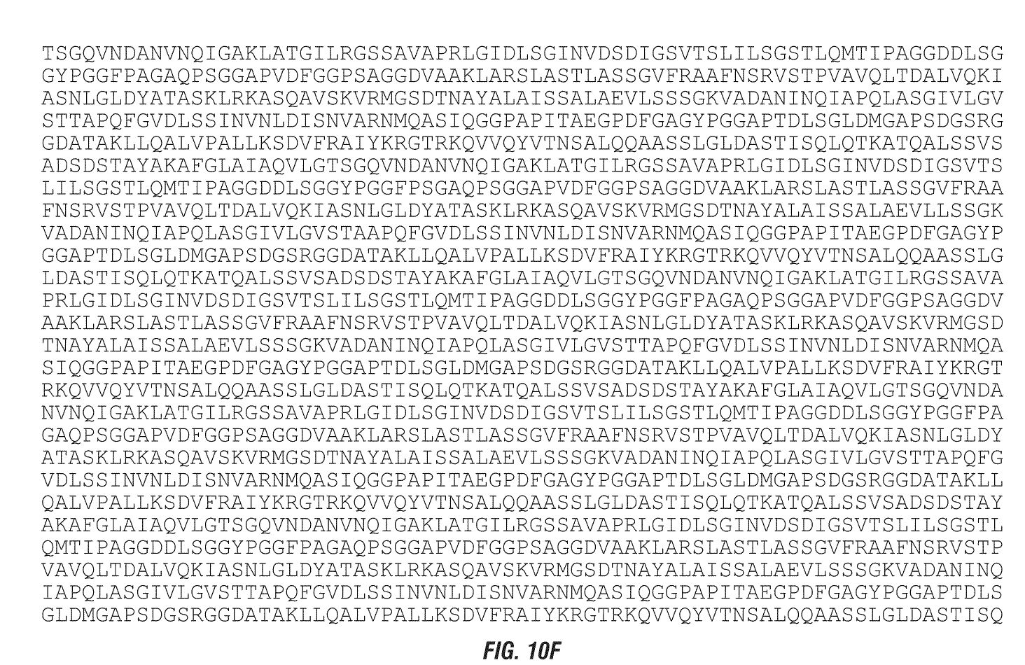

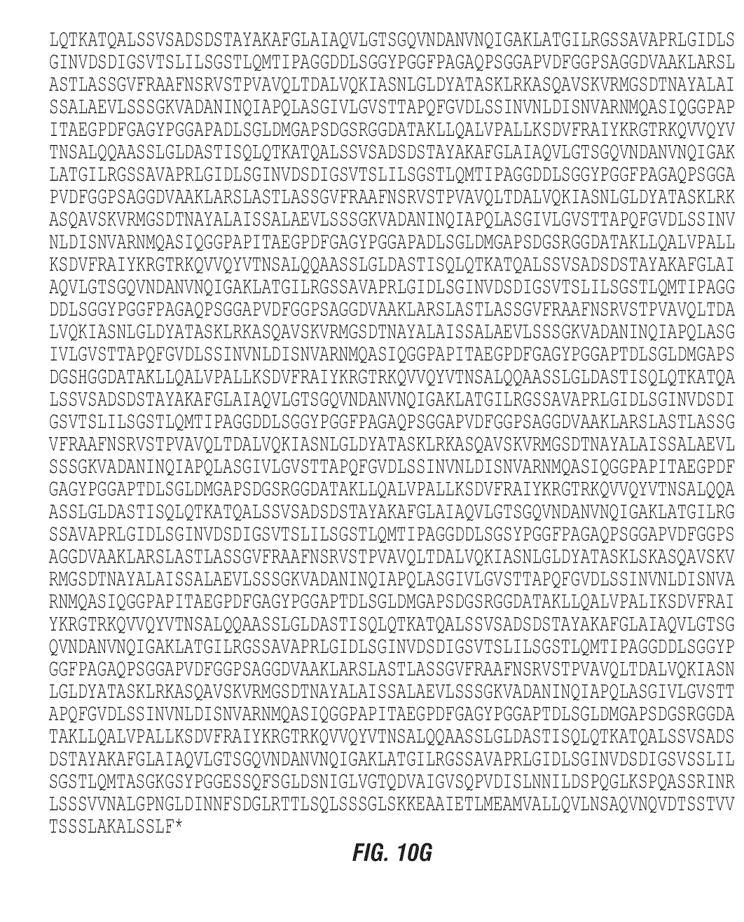

Likelihood and Bayesian analyses were also conducted with constraints placed for each gland-associated spidroin group (i.e., minor ampullate, major ampullate, flagelliform, tubuliform, pyriform, and aciniform gland types; FIG. 7; Table A). Our higher-level sampling was not intended to establish monophyly of each of the gland associated spidroin groups; rather the research aimed to determine the phylogenetic placements of the gland associated spidroin groups among spidroins from across the spider phylogeny. For minor ampullate, flagelliform, tubuliform, pyriform, and aciniform glands, spidroins have been reported from only a few species, while major ampullate spidroins are more widely known. The sample of major ampullate spidroins is not comprehensive because the research focused on sampling species for which multiple spidroins had been characterized. Using N and C-terminal sequences, Garb, Ayoub, and Hayashi (2010) recovered monophyletic groups for each of tubuliforms, flagelliforms, and minor ampullates in parsimony and Bayesian analyses. Entelegyne major ampullates spidroins were also recovered as monophyletic in their Bayesian analysis. N-terminal sequences have not been reported for aciniform and pyriform gland associated spidroins, or from any mygalomorph spidroins except for one (Bothriocyrtum californicum fib1). No N-terminal sequences in any of the libraries were recovered; thus this does not include published N-terminal sequences in the analyses. An SH test (Shimodaira and Hasegawa, 1999) using RAxML with the log likelihood values from the ML analyses was preformed to compare the constrained and unconstrained tree topologies.

The constrained ML spidroin gene tree was reconciled with a species tree based on Coddington et al. (2004), Hedin and Bond (2006), and Ayoub et al. (2007) using the program GeneTree 1.3 (Page, 1998). Spidroins lack a non-spider outgroup. Thus, rooting of the spidroin gene tree was based on the minimization of total gene duplications plus losses.

Characterization of Spidroin Non-Terminal Regions. Tandem repeats in spidroin protein sequences were identified using XSTREAM under default settings (Newman and Cooper, 2007). Consensus repeat sequences for each spidroin were determined based on 50% majority rule with ambiguities indicated by an X. Representative spidroin repeat lengths were based on repeat consensus lengths and published characterizations of repeats (Table S2). The amino acid compositions of spidroin repetitive regions was also determined with MacVector 7.2 (Accelrys Inc., San Diego, Calif.).

Using the ML tree from the analysis with gland associated spidroins constrained, continuous character, ancestral state reconstructions were performed for repeat length and amino acid compositions. Reconstructions were done using parsimony under the linear cost assumption in Mesquite v. 2.74 (Maddison and Maddison, 2010). Additionally, the Mesquite module, CoMET, was used to calculate the likelihood of observing the continuous data given the tree under nine different models of evolution (Lee et al., 2006). These models include pure phylogenetic, non-phylogenetic, or punctuated average, in combination with distance, equal, or free (Oakley et al., 2005). The best fitting model was determined by the Akaike Information Criterion (Akaike, 1973). CoMET analyses were run with thresholds of 100 and 1000 for comparison of the pure phylogenetic and punctuated average models. The punctuated average model was favored if the data was indicated to have evolved from branching events where the branch lengths were 100 or, more conservatively, 1000 times longer than their corresponding sister branch lengths (CoMET User's Guide, February 2006). Given that one of the newly characterized spidroins (Hypochilus fib2) and one previously characterized spidroin (Plectreurys fib3; Gatesy et al., 2001) each had two repeat motif types, CoMET analyses was performed using all four combinations of large and small motif lengths. Model choice in these various CoMET analyses was not influenced by these different inputs.

Liphistius Egg Case Protein Homologs.

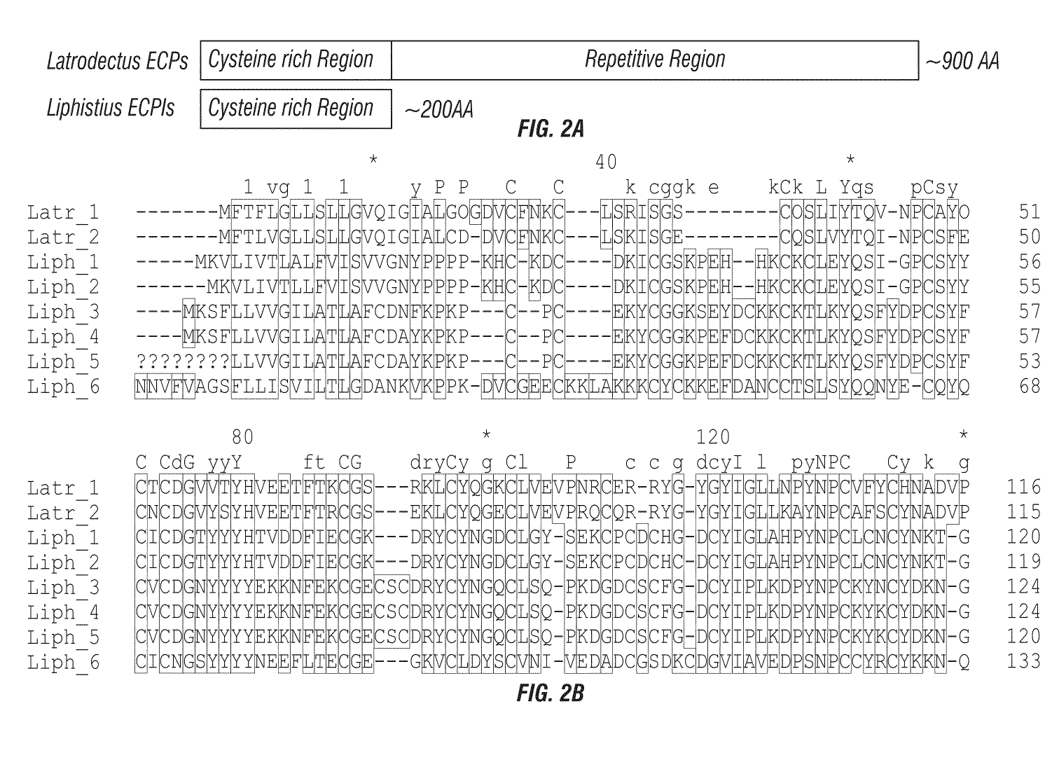

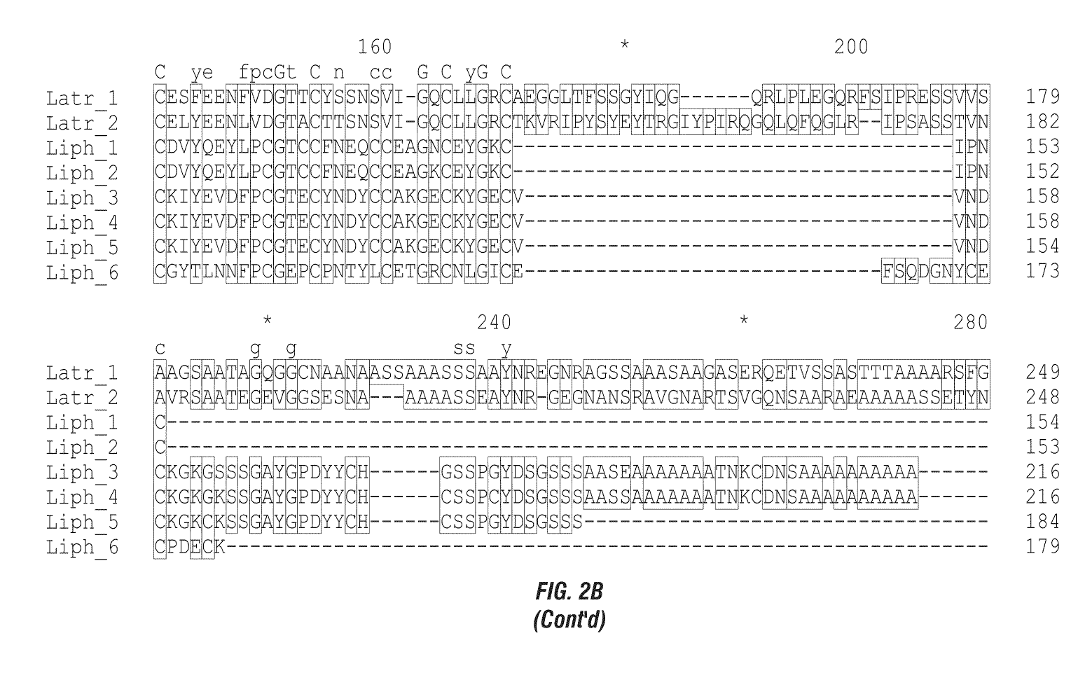

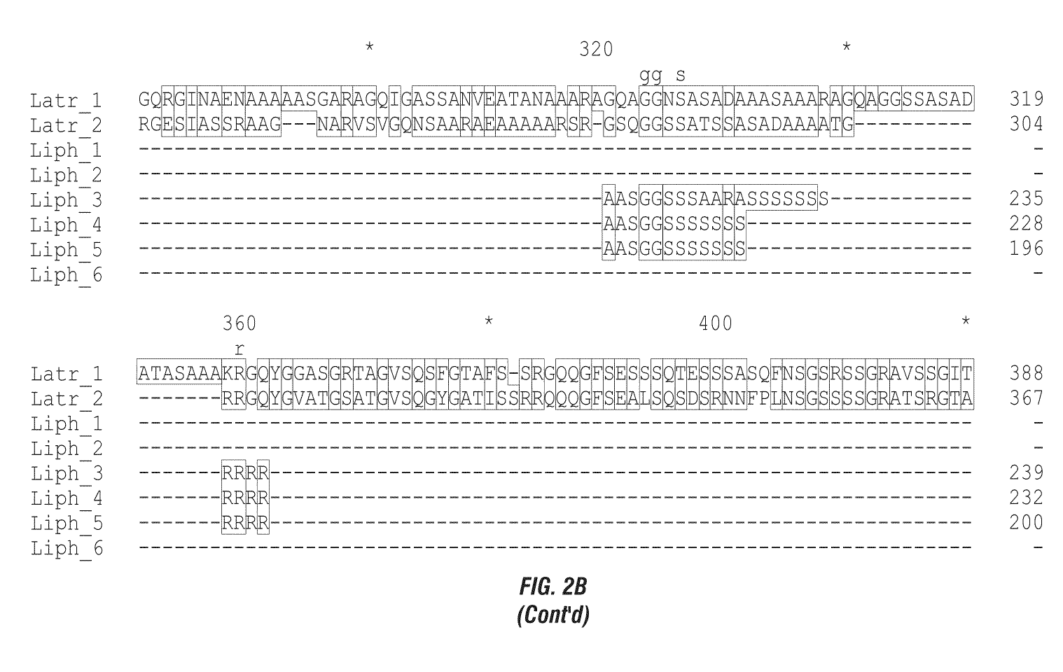

Blastx searches of cDNA clones identified six Liphistius transcripts with top hits to Latrodectus ECP1 (AY994149) and ECP2 (DQ341220). Thus, these Liphistius transcripts were named ECP-like (ECPL; GenBank accessions XXXX-YYYY). No ECP-like transcripts were detected in any of the mygalomorph cDNA libraries or the Hypochilus cDNA library. Liphistius ECPL names and cDNA lengths in base pairs (bp) in parentheses are as follows: ECPL1 (836), ECPL2 (724), ECPL3 (967), ECPL4 (969), ECPL5 (800), ECPL6 (950). With the exception of ECPL5, all of the Liphistius ECPL mRNA sequences included full length coding sequence. Liphistius ECPL transcripts are significantly shorter than Latrodectus ECP1 and ECP2 transcripts, which are 2799 by (coding, 932 AA) and 2478 bp (coding, 825 AA), respectively. The Liphistius ECPLs align to the non-repetitive, cysteine rich, N-terminal region, and lack most of the repetitive region of the Latrodectus ECPs (FIG. 2a). The average pairwise similarity for amino acid sequences (gaps treated as missing) among Liphistius ECPLs is 58.26%, and 33.53% between Liphistius ECPLs and Latrodectus ECPs (FIG. 2b).

Spidroin Gene Tree.

One or more spidroins were identified in the cDNA libraries for each taxon in our study, for a total of 13 new spidroins (GenBank accessions XXXX-YYYY). All of the spidroin cDNAs were partial length transcripts, lacking 5′ untranslated sequence, a start codon, N-terminal region sequence, and an unknown amount of repeat region sequence. Spidroin names with cDNA lengths (bp) in parentheses are as follows: Liphistius fib1 (3513); Hypochilus fib1 (2063) and fib2 (2190); Aphonopelma fib1 (1904), fib2 (1634), and fib3 (1464); Poecilotheria fib1 (4617) and fib2 (2437); Antrodiaetus fib1 (1833) and fib2 (5023); Sphodros fib1 (2460); and Hexura fib1 (409). Megahexura fib1 (1257) contained a C-terminal encoding region but lacked a complete repeat; therefore, an additional clone (4897) lacking C-terminal encoding region was sequenced. An overlapping region of these two clones confirmed that they likely represent the same transcript.

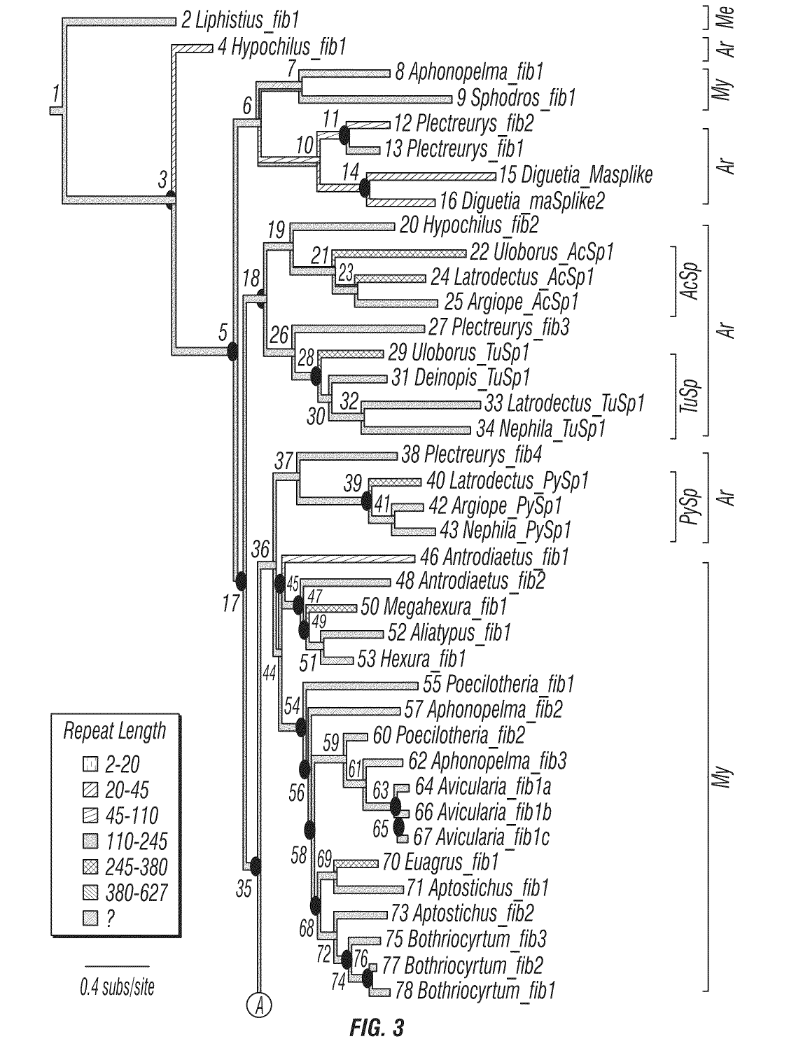

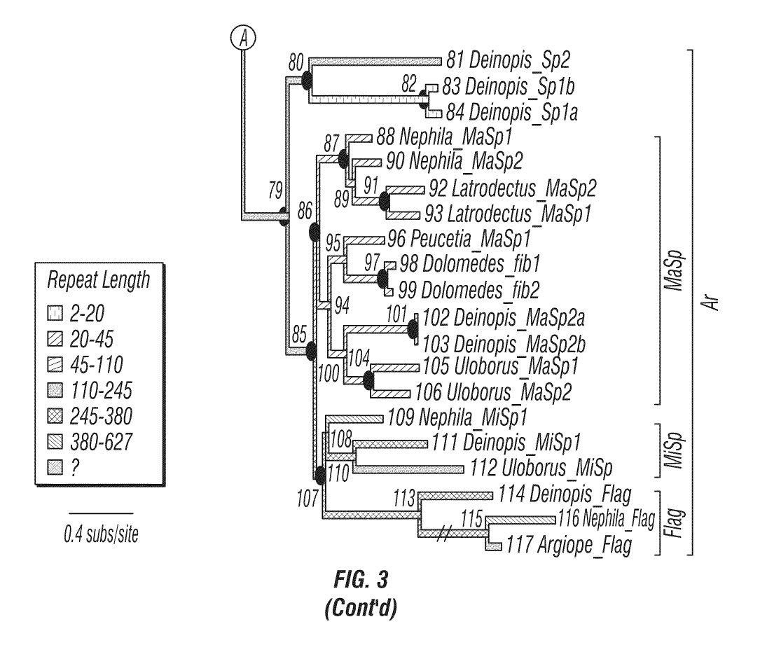

The tree based on the ML analysis with constraints was used (FIGS. 3, 7; Table A) for reconciliation analysis and reconstruction of the evolution of continuous characters (Table S2). While tubuliform, aciniform, pyriform, and flagelliform spidroins were each recovered as monophyletic in all ML and Bayesian analyses, without these constraints, monophyletic groupings of neither major ampullate spidroins nor minor ampullate spidroins were recovered. However, monophyly of both major ampullates and minor ampullates is supported by a previous Bayesian analysis of combined N and C-terminal data (Garb, Ayoub, and Hayashi, 2010). The ML constrained and unconstrained trees were identical at 46 of 58 nodes (FIG. 7). Conflicting relationships were restricted to weakly supported nodes (Table A). The SH (Shimodaira and Hasegawa, 1999) test determined that the constrained topology was not significantly worse than the unconstrained topology. Both the constrained and unconstrained Bayesian consensus trees were unresolved at many nodes (Table A). The ML and Bayesian constrained trees conflicted at only one node, where MiSps were placed sister to Flags in the ML analysis but sister to MaSps in the Bayesian analysis. The bootstrap and posterior probability were weak for either relationship.

The modest support at many nodes on the spidroin gene tree is not surprising given the small character set available (only C-terminal encoding regions) and the deep divergences among the taxa sampled. Support values for nodes of the spidroin gene tree will likely be improved in the future with inclusion of N-terminal regions, which are available for only a limited subset of published spidroins (Garb, Ayoub, and Hayashi, 2010). Our spidroin gene tree is generated from the broadest phylogenetic sampling of spider lineages to date and thus is the best available topology for reconciliation and ancestral character state reconstruction analyses.

Reconciliation analysis of the spidroin gene tree with the species tree supported the Liphistius spidroin as sister to all other spidroins (100 events=31 duplications+69 losses; FIG. 3; Table S2). Twenty-five other rootings implied the same number of duplications, but at an increased loss cost. Alternative rootings with Hypochilus fib1, or Hypochilus fib1 plus Liphistius fib1, resulted in the next best reconciliation score (101 events=31 duplications+70 losses) compared to the optimal score (rooting with Liphistius fib1).

Reciprocally monophyletic araneomorph and mygalomorph spidroin groups were never recovered in the phylogenetic analyses. Based on the most parsimonious rooting, Hypochilus fib1 was found to be sister to all remaining opisthothele spidroins, while Hypochilus fib2 was placed sister to the orbicularian aciniform spidroins (FIG. 3). Mygalomorph spidroins fell into two groups. The most basal mygalomorph group consisted of a tarantula spidroin (Aphonopelma fib1) and an atypoid spidroin (Sphodros fib1), and this Glade of genes was sister to spidroins from the haplogynes, Plectreurys and Diguetia. Most mygalomorph spidroins clustered in a group that was sister to an araneomorph Glade consisting of Plectreurys fib4 and all of the orbicularian pyriform spidroins. This second mygalomorph Glade is characterized by a basal split between atypoid spidroins and non-atypoid sequences; however, relationships within these two groups did not necessarily follow accepted species relationships (Hedin and Bond, 2006).

Spidroin Repeats.

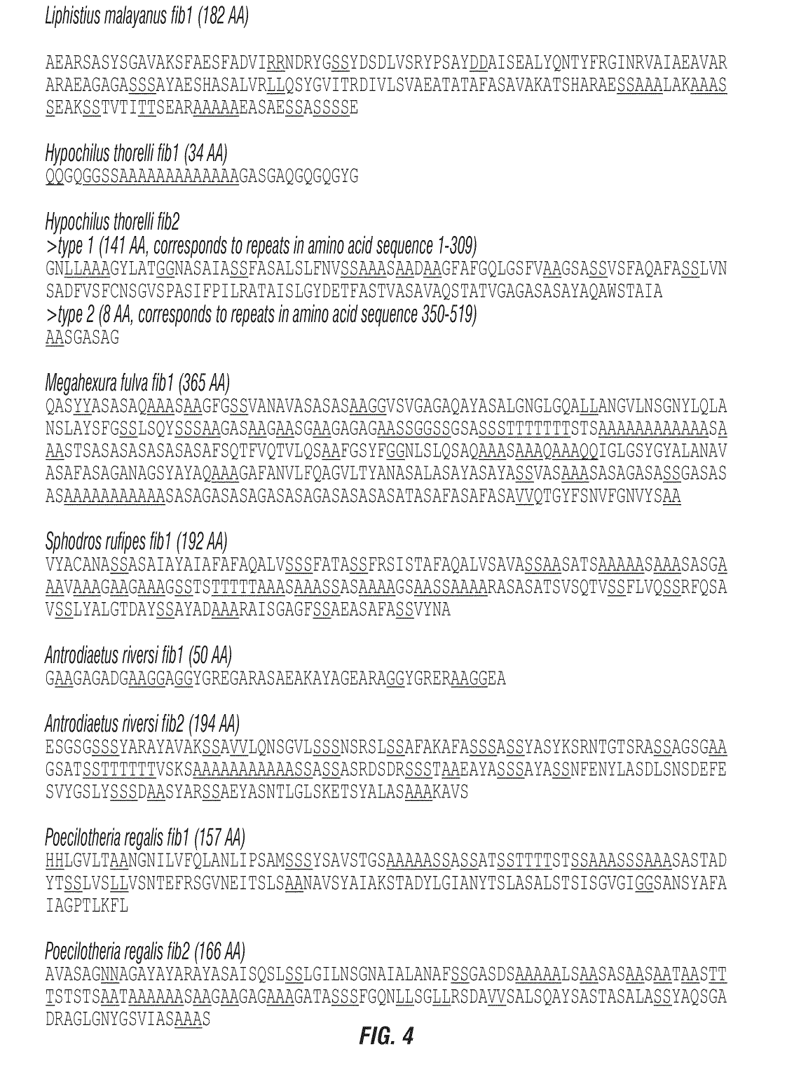

XSTREAM analyses identified repeat sequences in 9 of the 13 newly characterized spidroins (spidroin sequences Aphonopelma fib1, Aphonopelma fib2, Aphonopelma fib3 and Hexura fib1 were too short to record iterated repeats). Consensus repeats and their lengths are displayed in FIG. 4 and table S2. Most consensus repeat lengths are between 140 and 200 AA. Hypochilus fib1 and Antrodiaetus fib1 are significantly shorter at 34 and 50 AA, respectively. XSTREAM identified two repeat types in Hypochilus fib2. The consensus length of the first type, corresponding to repeats found in residues 1-309, is 141 AA. In contrast, the consensus repeat length of type two is 8 AA, and corresponds to repeats within residues 350-519. The Megahexura fib1 consensus repeat, at 365 AA, was significantly longer than the repeats from the other newly characterized spidroins described here. Unlike Euagrus fib1, which has a repeat of similar length (342 AA; Gatesy et al., 2001; Garb et al., 2007), the Megahexura fib1 repeat could not be broken down into sub-repeats of approximately ˜180 AA in length.

Parsimony ancestral state reconstruction of repeat length is shown in FIG. 3. Repeat length ranges for each node are given in table S2. The reconstructed ancestral condition for spidroins is the range 110-245 amino acids. Repeat lengths between 45 and 110 amino acids have evolved at least twice as seen in Plectreurys fib2 and Antrodiaetus fib1. Repeat lengths less than 45 amino acids have evolved convergently in Hypochilus fib1, Diguetia MaSp-likes, Deinopis fib1a and fib1b, and the major ampullates. Convergent evolution of repeat lengths greater than 245 has occurred in some aciniforms, Uloborus TuSp1, Latrodectus PySp1, Megahexura fib1, Euagrus fib1, some MiSps, and some Flags.

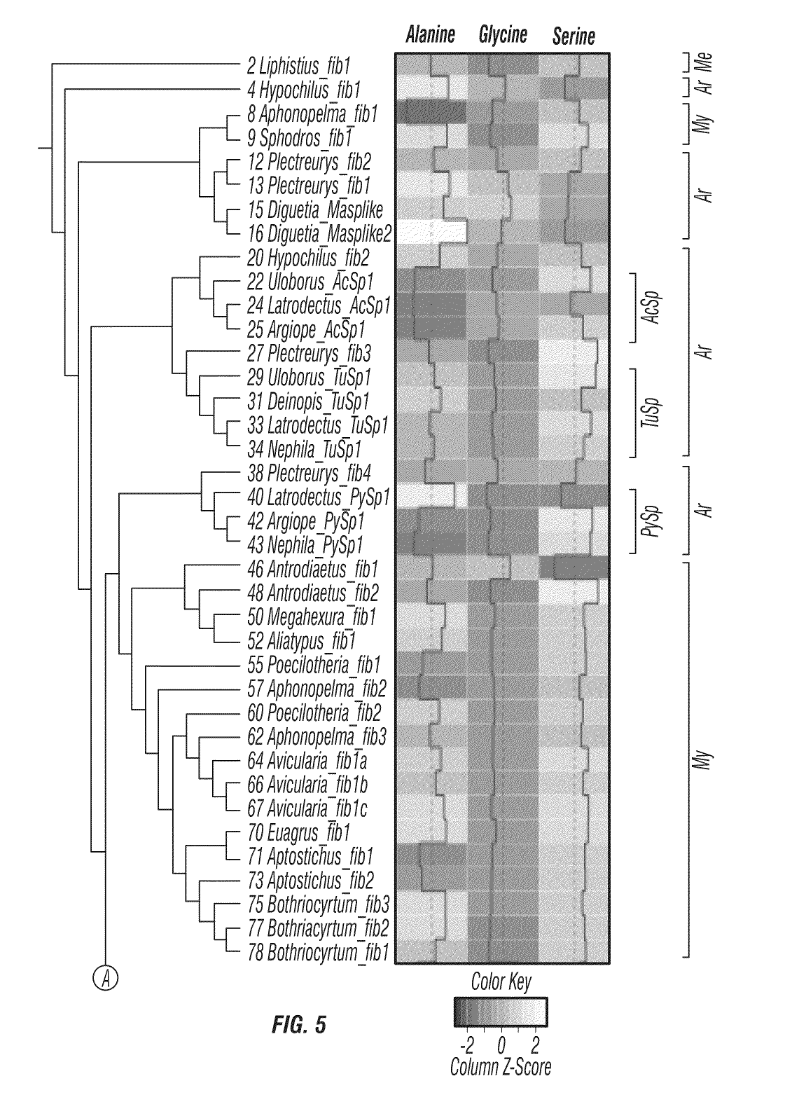

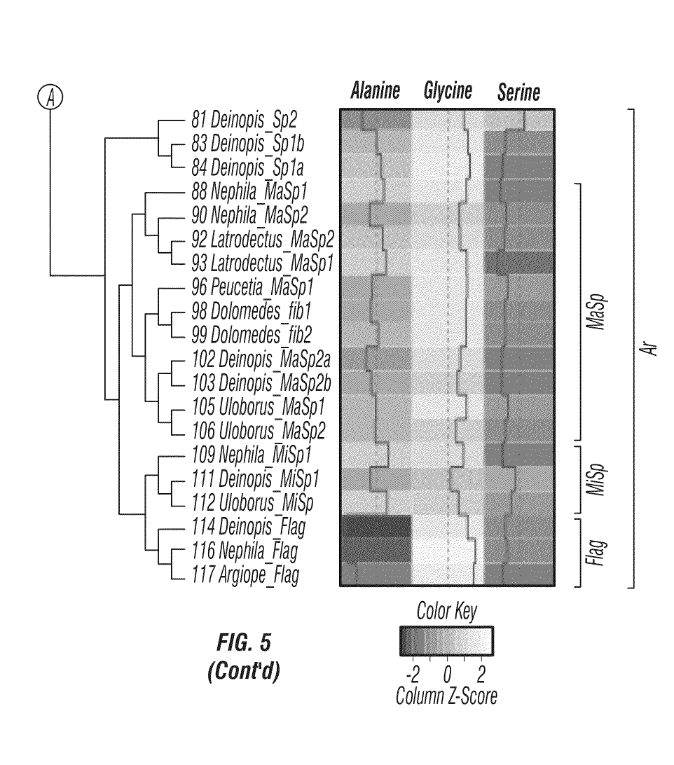

Repeat regions of most spidroins reported here are rich in alanine and serine, but low in glycine. Alanine and serine tandem repeats occur in all of the newly generated spidroin sequences, whereas iterations of other amino acids are less common (FIG. 4). The repeat region compositions of alanine, glycine, and serine for all spidroins analyzed in this study are summarized in table S2. The individual contributions of alanine, glycine, and serine relative to the total composition for each spidroin are displayed in a heat map (FIG. 5). Alanine levels are variable across spidroins. Glycine and serine levels appear to trade-off with each other in that they exhibit large and opposite changes. Glycine deficiency and high serine levels are primarily found in Liphistius, mygalomorph, and haplogyne spidroins, as well as tubuliform, aciniform, and pyriform gland-associated spidroins. By contrast, Deinopis fib1a and fib1b along with major ampullate, minor ampullate, and flagelliform gland-associated spidroins, have high glycine levels but are deficient in serine.

Continuous character modeling of amino acid composition and repeat lengths given our preferred tree (FIG. 3) were executed using CoMET, with optimal models chosen by the Akaike Information Criterion. For alanine composition, the Punctuated Average/Equal model was selected under the asymmetry threshold of 100, but the Pure-Phylogenetic/Distance model was selected under the asymmetry threshold of 1000. The Punctuated Average/Equal model was selected for glycine composition under thresholds of 100 and 1000. The model selected for serine composition was Pure-Phylogenetic/Distance under both thresholds. For all four combinations of repeat length, the Punctuated Average/Equal model was selected under thresholds of 100 and 1000.

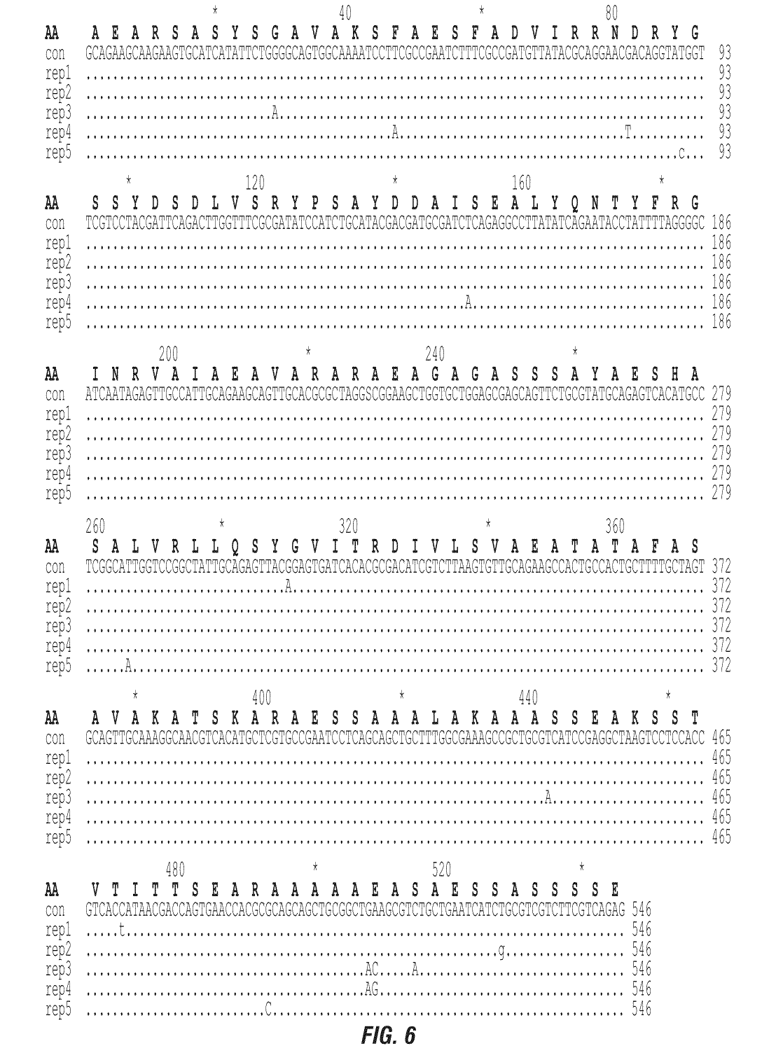

For each of the newly characterized spidroins, comparison of DNA sequences across repeats of a particular molecule reveal a high degree of sequence similarity among repeats. Hypochilus fib1, fib2 repeat 1, and fib2 repeat 2 showed the lowest average percent identities across repeat types at 85%, 79% and 77%, respectively. Repeats in the mygalomorph spidroin, Antrodiaetus fib1, shared 87% identity. Repeats within each of the six other new spidroins with identifiable repeats were >98% identical. A very low total of 13 non-synonymous differences and 3 synonymous differences occur across the 546 bp long alignment of Liphistius fib1 repeats (FIG. 6).

Liphistius Silk Gene Diversity.

The common ancestor of mesotheles and all other spiders is estimated to have existed more than 380 million years ago (Ayoub and Hayashi, 2009). This deep divergence and distant phylogenetic relationship with other spiders makes characterization of silk genes from Mesothelae crucial for obtaining a complete understanding of silk evolution. Mesotheles retain a number of plesiomorphic morphological characters associated with silk spinning (e.g., four pairs of anteriorally-placed spinnerets and single spigot types), and these spiders exhibit little variation in silk fiber types (Haupt and Kovoor, 1993; Haupt, 2003). However, mesotheles use silk for a variety of functions such as construction of their egg cases, burrow, trapdoor, and sensory lines (Shultz, 1987; Haupt and Kovoor, 1993). This combination of silk-spinning traits raises questions about the underlying diversity and function of silk genes and proteins from Mesothelae.

The Liphistius cDNA library included a considerable diversity of silk protein transcripts. In total, seven silk associated cDNAs were recovered, which approaches the number of different ortholog groups described from a single orb-weaver species and surpasses the number reported from most non-orbicularian araneomorph species (Blasingame et al., 2009; Gatesy et al., 2001; Garb, Ayoub, and Hayashi, 2010). This diversity is surprising given the much simpler silk gland morphologies of Liphistius compared to araneomorph spiders. Six of the seven Liphistius silk cDNAs shared substantial sequence similarity to the ECPs (egg case proteins; Blastx E values <1e-05), which have thus far only been reported from the Western black widow, Latrodectus hesperus (Hu et al., 2005a; Hu et al., 2006b). The six Liphistius egg case protein-like (ECPL) sequences group into three clusters. DNA sequence percent similarities across these three groups range from 49-57%. Within groups, percent similarities (gaps treated as missing) range from 96-100%. All of these sequences exhibit length differences in the protein-coding region, and for one of the groups, the only difference between members was a three-base pair indel. It is possible that some of the ECPL sequences represent allelic differences and/or splice variants.

The phylogenetic distribution of ECPs and ECPLs implies that egg case proteins either convergently evolved in Liphistius and Latrodectus, or that ECPs were present in the common ancestor of all extant spiders. Given the striking similarity of amino acids over a long region (˜200 residues) and lack of significant similarity to any other proteins in the NCBI nr database, it seems unlikely that ECPs evolved convergently in mesotheles and in theridiid araneomorphs (FIG. 2). Thus, we propose homology of Latrodectus ECPs and Liphistius ECPLs. However, a recent study on silk gland transcriptomes from the mygalomorph, Actinopus sp., and an orbicularian araneomorph, Gasteracantha cancriformis, also did not report ECPs (Prosdocimi et al., 2011). If our hypothesis of homology is correct, ECPs must have been lost independently in many spider lineages. Alternatively, ECPs may be highly restricted in their timing of expression, eluding detection in most cDNA libraries. With the completion of multiple spider genome sequences in the future, it will be possible to discern the presence, absence, or pseudogenization of ECPL genes in various spider taxa, and test the hypothesis of homology between the distantly related ECP and ECPL genes. In particular, synteny could provide additional evidence for orthology of ECPs from Latrodectus and ECPLs from Liphistius.

Both Latrodectus ECPs and Liphistius ECPLs are cysteine rich, with many cysteine positions conserved within and across species (FIG. 2; Hu et al., 2005a; Hu et al., 2006b). However, Liphistius ECPLs are significantly shorter than Latrodectus ECPs, lacking most of the extensive repetitive region seen in Latrodectus ECPs. While the timing and specificity of ECPL expression in Liphistius is uncertain, the physiochemical conservation of 73% of amino acids at sites that are present in at least one ECPL and ECP suggests that these ECPLs have a cross-linking role in silk fiber formation similar to that proposed for ECPs (Hu et al., 2006b).

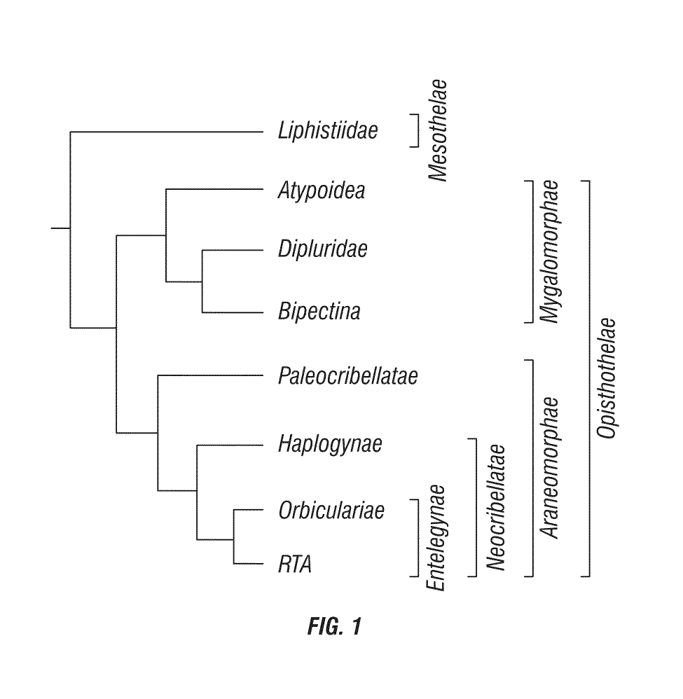

While mesotheles have high ECPL diversity, our cDNA screen suggests a low spidroin diversity, as only a single spidroin type (fib1) was detected in our Liphistius cDNA library. The presence of a spidroin in a mesothele confirms that the spidroin gene family evolved very early in Araneae and has an important role in silk production for all major spider groups that have been studied to date. Whether the Liphistius spidroin forms complexes with the ECPLs is currently unknown. In Latrodectus, ECPs form trimeric complexes with the N-terminal region of tubuliform spidroins (TuSp1) to make the outer silk wrapped around eggs (Hu et al., 2006b). The N-terminal region of Liphistius fib1 has not been characterized, but there are three cysteines in the C-terminal region that may allow for disulfide bonds with the ECPLs, as well as between fib1 monomers. Phylogenetic analyses did not recover a close relationship between Liphistius fib1 and TuSp1, indicating that TuSp1 is the result of spidroin duplication after the split of Opisthothelae from Mesothelae (FIGS. 1 and 3). This implies that ECPs evolved prior to TuSp1. Thus, ECPs likely first were incorporated into silk fibers made with spidroins that were serving a more general purpose, and later became incorporated into Latrodectus tubuliform silk fibers, which are specialized for egg case construction.

Spidroin Evolution.

The most parsimonious rooting of the spidroin gene tree using reconciliation analysis indicates that Liphistius fib1 is sister to all other spidroins (FIG. 3). Alternative less parsimonious rootings of the spidroin gene tree are consistent with spidroin gene family duplications occurring prior to the split of mesotheles and opisthotheles (Table S2). While mesotheles may have retained a single spidroin type, opisthotheles underwent an extensive diversification of spidroins very early in their history. Non-monophyly of araneomorph spidroins and of mygalomorph spidroins confirms that duplications occurred prior to the initial split of opisthotheles (Garb et al., 2007; Prosdocimi et al., 2011). The common ancestor of opisthotheles minimally had five spidroin paralogs (FIG. 3). These five paralogous gene lineages are now represented by 1) Hypochilus fib1, 2) a Glade consisting of two mygalomorph spidroins and four haplogyne spidroins, 3) a Glade consisting of orbicularian aciniform spidroins plus Hypochilus fib2 and orbicularian tubuliform spidroins plus Plectreurys fib3, 4) a Glade consisting of the remaining mygalomorph spidroins and orbicularian pyriform spidroins plus Plectreurys fib4, and 5) a Glade consisting of major and minor ampullates, orbicularian flagelliforms, and three additional Deinopis spidroins (FIG. 3).

The spidroin gene tree allows for inference of the duplication history of spidroins and how the origins of these different gene copies relate to the diversification of silk glands and to the evolution of spigot morphology. Mygalomorphs generally have a single spigot type and silk glands that are largely uniform and acinous in shape, which is thought to be the ancestral condition for spiders (Palmer, Coyle, and Harrison, 1982; Palmer, 1985; Shultz, 1987). Given the diversity of spidroins hypothesized in the opisthothele common ancestor, spidroin diversification preceded the evolution of morphologically distinct silk glands (FIG. 3).

The last common ancestor of araneomorphs is believed to have possessed ampullate, aciniform, pyriform, and cribellate silk glands and differentiated spigot types for each of these glands (Glatz, 1972; Coddington and Levi, 1991; Platnick et al., 1991). Spidroin ortholog groups associated with these glands are represented in the opisthothele common ancestor, with the exception of the cribellate spidroins, which to date have not been identified (FIG. 3). Additionally, tubuliform and aciniform spidroins are inferred to have resulted from gene duplication before the diversification of Araneomorphae. Tubuliform spigots are a synapomorphy for entelygyne araneomorphs, yet both tubuliform and aciniform spidroins have non-entelegyne relatives, consistent with spidroin diversification preceding the evolution of the morphologically distinct tubuliform gland and spigot type (Coddington, 1989; Platnick et al., 1991; Griswold et al., 1999). Based on our gene tree, the flagelliform, major ampullate, and minor ampullate spidroins appear to have diversified within Entelegynae. For the cDNA libraries from non-entelgyne spiders screened in this study, fibroins closely related to ampullate and flagelliform fibroins were not found.

No monophyly of mygalomorph spidroins were recovered in the phylogenetic analyses (Garb et al., 2007; Prosdocimi et al., 2011). In contrast to these other studies, our increased taxonomic sampling reveals that both of the mygalomorph spidroin clades include atypoid and non-atypoid spidroins, indicating that ancient spidroin duplicates may be retained in different mygalomorph taxa, as seen in Aphonopelma (FIG. 3). However, some mygalomorph taxa, such as Bothriocyrtum, retain spidroin copies that are very similar to each other, consistent with recent gene duplication or homogenization via concerted evolution in this mygalomorph lineage.

Mesothele and mygalomorph species have evolved a wide variety of web architectures, including sheet-webs, purse-webs, and trapdoors (Coyle, 1986). Assuming that the spidroins characterized from these taxa are those used to construct their webs, the relationship between different web shapes and the spidroins used to construct them appears to be highly variable. In many cases, closely related spidroin proteins may be used in the construction of very different web architectures. For example, Aliatypus spiders construct trapdoors, yet their spidroin is most closely related to the spidroins of Hexura and Megahexura, which construct sheet-webs (FIG. 3, FIG. 7). On the other hand, very similar architectures may be built from very divergent spidroins. Liphistius, Aliatypus, Aptostichus, and Bothriocyrtum have convergently evolved trapdoors, and the spidroins found from most of these spiders are not closely related. Thus, the ability of mesothele and mygalomorph species to produce different web architectures does not seem to be constrained by the silk proteins produced.

Evolution of Spidroin Repeat Regions. The analyses reveal very low nucleotide sequence variability among repeat units within a particular spidroin gene. Even Hypochilus fib2 and Plectreurys fib3, which are the only reported spidroins composed of two different repeat types, have high sequence similarity across repeats of the same type. Homogenization of repeats is consistent with concerted evolution via intragenic gene conversion or unequal crossing over, and is a pattern typical of spidroins reported from mygalomorph and araneomorph spiders (Hayashi and Lewis, 2000; Gatesy et al., 2001; Hayashi, Blackledge, and Lewis, 2004; Garb and Hayashi, 2005; Garb et al., 2007; Perry et al., 2010). The homogenization of repeats seen in Liphistius (FIG. 6) indicates that a gene architecture of tandemly arranged, homogenized repeats is an ancestral feature for spidroins.

The best fitting model determined by CoMET indicates that, on average, for branching events in the spidroin gene tree, one descendant spidroin retains the ancestral repeat length while the other descendant lineage diverges (Oakley et al., 2005). The pattern of punctuated change for repeat length across spidroins could be due to selection for divergent silk mechanical properties that are influenced by repeat motif structure. This divergence among spidroins for repeat length could be facilitated by concerted evolution, where a repeat unit is spread rapidly throughout a spidroin. Ancestral state reconstruction indicates that the archetypal repeat length is ˜180 amino acids (FIG. 3, Table S2). Liphistius fib1 and nearly all mygalomorph spidroin repeats are greater than 157 amino acids; an exception is Antrodiaetus fib1, which is significantly shorter at 50 amino acids in length. The large repeats seen in Euagrus (343 AA) and Megahexura (365 AA) resemble a multiple of the ˜180 AA unit. For example, Euagrus fib1 repeat can be divided into two subrepeats of approximately equal size that are 56% identical (Garb et al., 2007). Based on the alignment of subrepeats, it appears that the large size of the Euagrus fib1 repeat could be due to a concatenation of archetype-sized repeats. Further studies are needed to determine whether ˜180 amino acids is an optimal length for mygalomorph and mesothele silk production. At present, studies on recombinant silk production have focused on number of repeats and fiber formation, but not the influence of repeat size on fiber formation and mechanical properties (Brooks et al., 2008; An et al., 2011).

Alanine, glycine, and serine are three of the major amino acid components of spider silks and the silks of other arthropods (Hu et al., 2006a; Sutherland et al., 2010); for the spidroins analyzed here, these three amino acids account for, on average, 64% of the total amino acid content of the repetitive region. The percentages of these common amino acids vary considerably across the spidroin gene tree (FIG. 5, Table S2). For most spidroins, alanine levels fall within the range of 20-35%. This is also exhibited by the Liphistius spidroin (26.5% alanine), and ancestral state reconstruction posits 26-36% as the primitive condition for spider silks. The best fitting model for alanine under the most conservative asymmetry threshold in CoMET indicates that the sequence divergence level between spidroin C-terminal regions predicts the divergence level of alanine percentage in the repetitive regions (Oakley et al., 2005).

The heat map of the percent compositions of serine and glycine across the spidroin gene tree indicates that they contrast strikingly with each other (FIG. 5). Liphistius fib1, most myaglomorph spidroins, and most non-ampullate and non-flagelliform araneomorph spidroins exhibit moderately high serine levels, but are deficient in glycine. In contrast, ampullate and flagelliform spidroins show high levels of glycine and low levels of serine. The best fitting CoMET model determined for glycine percentage suggests that at branching events in the spidroin gene tree, one spidroin retains the ancestral glycine level while the other descendant gene lineage diverges (Oakley et al., 2005). Punctuated evolution of glycine could be due to selection for sequence encoding glycine rich motifs, spread rapidly throughout the gene by concerted evolution, and maintained thereafter by stabilizing selection. Glycine rich motifs are known to contribute to the high tensile strength and extensibility of major ampullate and flagelliform silk fibers, respectively (Hu et al., 2006a). As was the case for alanine, the best fitting model selected for serine percentage indicates that change in serine composition more closely reflects the level of spidroin C-terminal sequence divergence (Oakley et al., 2005). Therefore, while alanine and serine percentages change across spidroins, they do not exhibit the pattern of large change followed by stasis to the extent that is seen in glycine levels.

Spider silks vary greatly in mechanical performance across species and among silks associated with different gland types (Swanson et al., 2006; Blackledge and Hayashi, 2006; Boutry and Blackledge, 2010). Tensile testing of silks from representatives of Liphistius and mygalomorphs has shown that these silks have lower tensile strength than major ampullate silks and lack the high extensibility of flagelliform silks (Swanson et al., 2009; Blackledge and Hayashi, 2006). Thus far, silk mechanical properties have only been tested on a few mesothele species and theraphosid mygalomorphs (tarantulas). Our study reveals mygalomorph silk proteins with distinct molecular architectures that may enable unique, and perhaps exceptional, mechanical properties. For example, Antrodiaetus expresses two silk encoding genes, one of which (fib1) encodes a protein with a glycine percentage of ˜30%, which is more comparable to major ampullate and minor ampullate silks (˜24-45%) than theraphosid silks (<10%). Also, the repeat length encoded by Megahexura fib1 is −365 amino acids, which is well above the known range of repeat lengths encoded by theraphosid spidroin genes (157-186 amino acids). Thus, broader examination of silk mechanical properties in different mygalomorphs is warranted.

Mesotheles and mygalomorphs mostly use their silks to line their burrows, construct retreats, make egg sacs, and extend their sensory area. Exceptionally extensible or strong silk may not be advantageous for these purposes (Coyle, 1986). These spiders rely on their size, power, and robust fangs to capture ground dwelling prey, and there is little need for silks capable of absorbing kinetic energy from flying insects. Instead, selection in mesothele and mygalomorph lineages may favor durable silks that are optimized for stability in subterranean conditions or for sensitivity in detection of vibrations from prey. The new silk genes found can be used to further investigate silk mechanical and functional properties and how these relate to the subterranean lifestyle of mesotheles and mygalomorphs.

Analysis of silk gland expression libraries from mesothele, paleocribellate, and mygalomorph spiders greatly clarifies the evolutionary history of silk in Araneae. The discovery of mesothele ECPL sequences that share conserved regions with Latrodectus ECPs suggests that these loci comprise a gene family which has been associated with silk production in spiders for >380 million years. Further research is needed to determine the phylogenetic breadth of this gene family in spiders, as well as how ECPs functionally interact with members of the spidroin gene family. Phylogenetic analysis of our new data from Mesothelae, Mygalomorphae, and Hypochilus suggests that the most recent common ancestor of all extant spiders had a single spidroin, and that diversification of spidroins by gene duplication had already occurred prior to the divergence of mygalomorphs and araneomorphs. The repeat regions vary considerably in structure and amino acid composition across different spidroin types. The punctuated pattern of change in repeat length and glycine percentage could be due to selection for improved mechanical properties enabled by these characteristics, facilitated by concerted evolution quickly spreading desirable protein coding motifs throughout a spidroin gene.

Mesotheles and mygalomorphs construct a wide variety of web shapes and burrow entrance architectures. Considering the ecological function of mygalomorph and mesothele silks, selection on silk from these spiders may have favored properties associated with the largely subterranean niche they fill, such as durability for burrow maintenance and vibration transmission for prey capture (Coyle, 1986). The diversity of silk genes uncovered in mesotheles and mygalomorphs highlights the need for further exploration into the phylogenetic diversity of spiders for silk genes that encode unique silk mechanical properties.

TABLE A
Node support (ML bootstrap and Bayesian posterior probabilities) for
phylogenetic analyses. Node numbers refer to the phylogeny in FIGS.
3 and 7. Dashes refer to nodes with <50 BS or 0.5 PP support.

| | |
|---|---|
| ML | Bayes |

| | Con- | Uncon- | Con- | Uncon- |
|---|---|---|---|---|
| Node# | strained (C) | strained | strained (C) | strained |
| 1 | — | — | — | — |
| 3 | — | — | — | — |
| 5 | — | — | 0.78 | 0.8 |
| 6 | — | — | — | — |
| 7 | 65 | 67 | 0.97 | 0.98 |
| 10 | 83 | 79 | 1.0 | 0.99 |
| 11 | 92 | 89 | 1.0 | 1.0 |
| 14 | 87 | 87 | 1.0 | 1.0 |
| 17 | — | — | — | — |
| 18 | — | — | — | — |
| 19 | — | — | 0.57 | 0.66 |
| 21 | C, 97 | 90 | C, 1.0 | 1.0 |
| 23 | — | 53 | — | 0.56 |
| 26 | — | — | 0.54 | 0.5 |
| 28 | C, 74 | — | C, 1.0 | 0.97 |
| 30 | — | — | 0.51 | 0.53 |
| 32 | 68 | 56 | 0.94 | 0.93 |
| 35 | — | — | 0.74 | 0.77 |
| 36 | — | — | — | — |
| 37 | — | — | 0.59 | 0.62 |
| 39 | C, 94 | 92 | C, 1.0 | 1.0 |
| 41 | 75 | 75 | 0.94 | 0.95 |
| 44 | — | — | — | — |
| 45 | — | — | — | — |
| 47 | — | — | 0.65 | 0.59 |
| 49 | — | — | — | — |
| 51 | 55 | 52 | 0.91 | 0.9 |
| 54 | — | — | 0.67 | 0.67 |
| 56 | — | — | — | 0.5 |
| 58 | — | — | — | — |
| 59 | 82 | 82 | 1.0 | 1.0 |
| 61 | 83 | 80 | 1.0 | 1.0 |
| 63 | 93 | 93 | 1.0 | 1.0 |
| 65 | 56 | — | — | — |
| 68 | — | — | — | — |
| 69 | 70 | 72 | 0.93 | 0.93 |
| 72 | — | — | 0.94 | 0.95 |
| 74 | 59 | 54 | 0.97 | 0.97 |
| 76 | 98 | 97 | 1.0 | 1.0 |
| 79 | — | — | 0.98 | 0.98 |
| 80 | — | — | 0.84 | 0.61 |
| 82 | 100 | 100 | 1.0 | 1.0 |
| 85 | — | — | 0.61 | 0.5 |
| 86 | C, — | — | C, 1.0 | — |
| 87 | 82 | 69 | 1.0 | 0.99 |
| 89 | 67 | 61 | 0.81 | 0.82 |
| 91 | 98 | 98 | 1.0 | 1.0 |
| 94 | — | — | 0.94 | 0.71 |
| 95 | 63 | — | 0.99 | 0.94 |
| 97 | 95 | 94 | 1.0 | 1.0 |
| 100 | 82 | 77 | 0.96 | 0.96 |
| 101 | 100 | 100 | 1.0 | 1.0 |
| 104 | 99 | 99 | 1.0 | 1.0 |
| 107 | — | — | — | — |
| 108 | C, 55 | — | C, 1.0 | — |
| 110 | — | — | 0.69 | — |
| 113 | C, 91 | — | C, 1.0 | 0.99 |
| 115 | 100 | 100 | 1.0 | 1.0 |

TABLE B
Continuous character data and alternative
reconciliation based outgroups for ML constrained tree.
Citations indicate source of repeat length characterization.

| | | | | | | Alternative |
|---|---|---|---|---|---|---|
| | | | | | | Rooting/ |
| | | | | | | Outgroup |
| | | | | Repeat | | duplication/ |
| | | | | Length | | loss/deep |
| | | | | (long/short | Repeat Length | coalescence |
| Node # | Alanine % | Glycine % | Serine % | combinations) | Characterization | events |
| 1 | 26.5-36.44 | 3.15-12.83 | 17.18-19.03 | 180-182/179-182 | | |
| 2 (Liphistius_fib1) | 26.5 | 3.15 | 19.03 | 182 | This study | 31/69/81 |
| 3 | 26.5-36.44 | 9.69-12.83 | 17.18-18.94 | 180-182/179-180 | | |
| 4 (Hypochilus_fib1) | 40.28 | 23.7 | 9.72 | 34 | This study | 31/70/82 |
| 5 | 26.5-36.44 | 9.69-12.83 | 17.18-18.94 | 180-182/179-180 | | 31/70/82 |
| 6 | 26.5-36.44 | 9.69-12.83 | 17.18-18.94 | 180-182/179-180 | | 31/71/83 |
| 7 | 26.5-36.44 | 7.13 | 18.94 | ? | | |
| 8 (Aphonopelma_fib1) | 8.15 | 7.13 | 18.94 | ? | This study | |
| 9 (Sphodros_fib1) | 39.07 | 4.52 | 24.78 | 192 | This study | |
| 10 | 29.1-36.44 | 13.15-19.0 | 12.64-18.94 | 66-126 | | |
| 11 | 29.1-36.44 | 13.15-19.0 | 12.64-18.94 | 66-126 | | |
| 12 (Plectreurys_fib2) | 29.1 | 13.15 | 20.09 | 66 | Gatesy et al. | |
| | | | | | (2001) | |
| 13 (Plectreurys_fib1) | 41.19 | 27.1 | 12.64 | 126 | Gatesy et al. | |
| | | | | | (2001) | |
| 14 | 36.44 | 19 | 11.74 | 39 | | |
| 15 | 36.44 | 30.97 | 11.74 | 39 | Garb, Ayoub, | |
| (Diguetia_MaSplike) | | | | | and Hayashi | |
| | | | | | (2010) | |
| 16 | 54.75 | 19 | 9.95 | 24 | Garb, Ayoub, | |
| (Diguetia_MaSplike2) | | | | | and Hayashi | |
| | | | | | (2010) | |
| 17 | 25.26-29.65 | 9.69-12.83 | 17.18-18.94 | 180-182/179-180 | | 31/71/83 |
| 18 | 25.26-29.65 | 9.69-12.83 | 20.53-21.46 | 180-206/179-180 | | 31/72/84 |
| 19 | 25.26-29.65 | 9.69-12.83 | 20.53-21.46 | 180-206/179-180 | | 31/74/86 |
| 20 (Hypochilus_fib2) | 33.68 | 13.51 | 20.53 | 141/8 | This study | |
| 21 | 14.67 | 9.69-12.83 | 20.53-21.46 | 200-357 | | |
| 22 (Uloborus_AcSp1) | 14.67 | 7.29 | 26.63 | 357 | Garb et al. | |
| | | | | | (2006) | |
| 23 | 14.15 | 12.83 | 20.53-21.46 | 200-357 | | |
| 24 | 13.43 | 12.83 | 13.63 | 376 | Vasanthavada et | |
| (Latrodectus_AcSp1) | | | | | al. (2007) | |
| 25 (Argiope_AcSp1) | 14.15 | 15.5 | 21.46 | 200 | Hayashi, | |
| | | | | | Blackledge, and | |
| | | | | | Lewis (2004) | |
| 26 | 25.26-29.65 | 9.69 | 22.11-29.91 | 200-206/179-180 | | 31/74/86 |
| 27 (Plectreurys_fib3) | 25.26 | 2.14 | 30.94 | 206/46 | Gatesy et al. | |
| | | | | | (2001) | |
| 28 | 29.21-29.65 | 9.69 | 22.11-29.91 | 200-206/184-200 | | |
| 29 (Uloborus_TuSp1) | 29.65 | 9.69 | 29.91 | 302 | Garb et al. | |
| | | | | | (2006) | |
| 30 | 29.21-29.65 | 9.69 | 22.11-26.86 | 200/184-200 | | |
| 31 (Deinopis_TuSp1) | 34.5 | 11.78 | 20.34 | 200 | Garb et al. | |
| | | | | | (2006) | |
| 32 | 29.21 | 8.52 | 22.11-26.86 | 184 | | |
| 33 | 26.01 | 6.75 | 26.86 | 184 | Garb et al. | |
| (Latrodectus_TuSp1) | | | | | (2007) | |
| 34 (Nephila_TuSp1) | 29.21 | 8.52 | 22.11 | 171 | Tian and Lewis | |
| | | | | | (2005) | |
| 35 | 24.23-27.59 | 9.69-12.83 | 17.18-18.94 | 180-182/179-180 | | 31/72/84 |
| 36 | 22.44-27.59 | 8.25-12.83 | 17.18-18.94 | 180-182/179-180 | | 31/73/85 |
| 37 | 22.44-27.59 | 8.25-12.83 | 17.18-18.94 | 226-239 | | |
| 38 (Plectreurys_fib4) | 22.44 | 13.85 | 17.18 | 239 | Gatesy et al. | |
| | | | | | (2001) | |
| 39 | 22.44-27.59 | 1.97-5.41 | 17.18-18.94 | 226-239 | | |
| 40 | 45.15 | 0 | 7.26 | 316 | Blasingame et | |
| (Latrodectus_PySp1) | | | | | al. (2009) | |
| 41 | 17.69 | 1.97-5.41 | 26.6 | ? | | |
| 42 (Argiope_PySp1) | 17.69 | 5.41 | 27.76 | ? | Perry et al. | |
| | | | | | (2010) | |
| 43 (Nephila_PySp1) | 13.79 | 1.97 | 26.6 | 226 | Perry et al. | |
| | | | | | (2010) | |
| 44 | 22.44-27.59 | 8.25-12.83 | 17.18-21.38 | 179-182/179-180 | | |
| 45 | 23.9-28.39 | 8.25-12.83 | 17.18-21.38 | 179-182/179-180 | | |
| 46 (Antrodiaetus_fib1) | 28.39 | 29.66 | 2.97 | 50 | This study | |
| 47 | 23.9-28.39 | 6.23-10.53 | 22.09-23.07 | 181-194 | | |
| 48 (Antrodiaetus_fib2) | 23.9 | 4.92 | 31.45 | 194 | This study | |
| 49 | 35.17 | 6.23-10.53 | 22.09-23.07 | 181-194 | | |
| 50 (Megahexura_fib1) | 38.07 | 10.53 | 22.09 | 365 | This study | |
| 51 | ? | ? | ? | ? | | |
| 52 (Aliatypus_fib1) | 35.17 | 6.23 | 23.07 | 181 | Garb et al. | |
| | | | | | (2007) | |
| 53 (Hexura_fib1) | ? | ? | ? | ? | This study | |
| 54 | 20.7-27.59 | 8.25-8.9 | 19.37-21.38 | 179 | | |
| 55 (Poecilotheria_fib1) | 20.7 | 6.46 | 23.57 | 157 | This study | |
| 56 | 20.7-27.59 | 8.25-8.9 | 19.37-21.38 | ? | | |
| 57 (Aphonopelma_fib2) | 17.8 | 8.9 | 19.37 | ? | This study | |
| 58 | 20.7-33.23 | 8.25-8.9 | 21.38 | 179 | | |
| 59 | 26.03-33.23 | 8.25-8.9 | 21.38 | 179 | | |
| 60 (Poecilotheria_fib2) | 33.23 | 10.15 | 21.38 | 166 | This study | |
| 61 | 26.03-33.23 | 8.25 | 21.38 | ? | | |
| 62 (Aphonopelma_fib3) | 26.03 | 8.25 | 20.32 | ? | This study | |
| 63 | 30.65-34.51 | 8.24-8.25 | 21.74-24.9 | 179-183 | | |
| 64 (Avicularia_fib1a) | 34.51 | 8.24 | 24.9 | 183 | Bittencourt et | |
| | | | | | al. (2010) | |
| 65 | 30.65-34.51 | 8.24-8.25 | 21.74-24.9 | 179-183 | | |
| 66 (Avicularia_fib1b) | 30.65 | 9.57 | 21.74 | 177 | Bittencourt et | |
| | | | | | al. (2010) | |
| 67 (Avicularia_fib1c) | 36.95 | 5.99 | 25.2 | 186 | Bittencourt et | |
| | | | | | al. (2010) | |
| 68 | 20.7-33.23 | 6.98-8.9 | 22.58-22.81 | 179 | | |
| 69 | 20.7-33.23 | 6.98-8.9 | 22.81 | 179 | | |
| 70 (Euagrus_fib1) | 38.57 | 11.75 | 24.92 | 343 | Gatesy et al. | |
| | | | | | (2001) | |
| 71 (Aptostichus_fib1) | 18.02 | 6.98 | 22.81 | 169 | Garb et al. | |
| | | | | | (2007) | |
| 72 | 20.7-33.23 | 6.07-6.19 | 22.58-22.81 | 179 | | |
| 73 (Aptostichus_fib2) | 19.97 | 6.07 | 23 | 179 | Garb et al. | |
| | | | | | (2007) | |
| 74 | 30.43-38.25 | 6.07-6.19 | 22.58-22.81 | 179 | | |
| 75 | 38.25 | 6.19 | 21.04 | 183 | Garb et al. | |
| (Bothriocyrtum_fib3) | | | | | (2007) | |
| 76 | 30.43-38.25 | 4.66 | 22.58-22.81 | 175 | | |
| 77 | 38.28 | 4.66 | 22.58 | 175 | Garb et al. | |
| (Bothriocyrtum_fib2) | | | | | (2007) | |
| 78 | 30.43 | 3.52 | 23.4 | 171 | Garb et al. | |
| (Bothriocyrtum_fib1) | | | | | (2007) | |
| 79 | 24.23-27.59 | 40.22-40.91 | 6.9-18.94 | 180-182/179-180 | | 31/73/85 |
| 80 | 24.23-27.59 | 40.91 | 6.9-18.94 | 180/179-180 | | 31/77/89 |
| 81 (Deinopis_fib2) | 16.48 | 40.91 | 19.13 | 180 | Garb et al. | 31/84/96 |
| | | | | | (2006) | |
| 82 | 27.59 | 46.06 | 6.9 | 2 | | 31/84/96 |
| 83 (Deinopis_fib1b) | 27.59 | 46.06 | 6.9 | 2 | Garb et al. | 31/91/103 |
| | | | | | (2006) | |
| 84 (Deinopis_fib1a) | 31.14 | 48.23 | 4.86 | 2 | Garb et al. | 31/91/103 |
| | | | | | (2006) | |
| 85 | 24.23-27.59 | 40.22-40.91 | 6.7-6.73 | 180-182/179-180 | | 31/77/89 |
| 86 | 24.23-27.59 | 40.22-40.91 | 6.7-6.73 | 33-34 | | 31/81/93 |
| 87 | 24.23-32.25 | 40.22-40.91 | 6.12-6.73 | 33-34 | | 31/85/97 |
| 88 (Nephila_MaSp1) | 33.1 | 44.13 | 3.56 | 30 | Gatesy et al. | 31/92/104 |
| | | | | | (2001) | |
| 89 | 24.23-32.25 | 35.83-40.91 | 6.12-6.73 | 33-34 | | 31/92/104 |
| 90 (Nephila_MaSp2) | 22.26 | 35.09 | 7.55 | 40 | Gatesy et al. | |
| | | | | | (2001) | |
| 91 | 32.25 | 35.83-40.91 | 6.12 | 33-34 | | |
| 92 | 32.25 | 35.83 | 6.12 | 24 | Garb et al. | |
| (Latrodectus_MaSp2) | | | | | (2007) | |
| 93 | 34.85 | 44.47 | 1.86 | 34 | Garb et al. | |
| (Latrodectus_MaSp1) | | | | | (2007) | |
| 94 | 24.23-26.5 | 40.22-40.91 | 6.7-6.73 | 33-34 | | 31/85/97 |
| 95 | 24.23-26.5 | 43.55-43.6 | 6.7-6.73 | 33 | | |
| 96 (Peucetia_MaSp1) | 24.23 | 44.9 | 8.42 | 32 | Pérez-Rigueiro | |
| | | | | | et al. (2010) | |
| 97 | 24.23-26.5 | 43.55-43.6 | 6.7-6.73 | 33 | | |
| 98 (Dolomedes_fib1) | 23.12 | 43.55 | 6.51 | 33 | Gatesy et al. | |
| | | | | | (2001) | |
| 99 (Dolomedes_fib2) | 28.96 | 43.6 | 6.73 | 33 | Gatesy et al. | |
| | | | | | (2001) | |
| 100 | 24.23-26.5 | 36.22-40.91 | 6.7-6.73 | 33-34 | | |
| 101 | 23.61 | 36.22 | 4.46 | 33-34 | | |
| 102 | 19.41 | 36.22 | 4.45 | 38 | Garb et al. | |
| (Deinopis_MaSp2a) | | | | | (2006) | |
| 103 | 23.61 | 32.97 | 4.46 | 32 | Garb et al. | |
| (Deinopis_MaSp2b) | | | | | (2006) | |
| 104 | 26.5 | 36.22-40.91 | 6.84 | 33-34 | | |
| 105 (Uloborus_MaSp1) | 26.92 | 44.23 | 8.65 | 36 | Garb et al. | |
| | | | | | (2006) | |
| 106 (Uloborus_MaSp2) | 26.5 | 30.77 | 6.84 | 28 | Garb et al. | |
| | | | | | (2006) | |
| 107 | 24.23-27.59 | 40.22-40.91 | 6.7-6.73 | 321 | | 31/81/93 |
| 108 | 24.23-35.47 | 40.22 | 6.7-6.73 | 321-335 | | 31/86/98 |
| 109 (Nephila_MiSp1) | 36.52 | 40.22 | 4.83 | 627 | Gatesy et al. | |
| | | | | | (2001) | |
| 110 | 24.23-35.47 | 34.16 | 10.73 | 321-335 | | |
| 111 (Deinopis_MiSp1) | 22.83 | 24.07 | 13.4 | 335 | Garb et al. | |
| | | | | | (2006) | |
| 112 (Uloborus_MiSp) | 35.47 | 34.16 | 10.73 | 147 | Garb et al. | |
| | | | | | (2006) | |
| 113 | 5.49-12.01 | 45.58 | 6.7-6.73 | 321 | | 31/86/98 |
| 114 (Deinopis_Flag) | 3.49 | 45.58 | 6.7 | 321 | Garb et al. | |
| | | | | | (2006) | |
| 115 | 5.49-12.01 | 53 | 6.7-6.73 | 321 | | |
| 116 (Nephila_Flag) | 5.49 | 55.24 | 8.05 | 420 | Gatesy et al. | |
| | | | | | (2001) | |
| 117 (Argiope_Flag) | 12.01 | 53 | 5.3 | 134 | Gatesy et al. | |
| | | | | | (2001) | |

A number of non-limiting examples and embodiment have been described. The foregoing description is not intended to limit the invention and one of skill in the art will readily ascertain additional embodiments encompassed by the following claims in view of the foregoing description.

1. A substantially purified polypeptide comprising an N-terminus sequence that is about 80-150 amino acids in length and having at least about 90% identity to SEQ ID NO:5 from amino acid position 71 to 151 followed by a sequence of SEQ ID NO:5 from position 1358-1384, wherein SEQ ID NO:5 from position 1358-1384 is preceded and/or followed by a glycine and alanine rich region and having a C-terminal region of about 50-100 amino acids in length and having at least about 90% identity to SEQ ID NO:5 from position 2085 to 2141, wherein the polypeptide has a property selected from the group consisting of (i) a Young's modulus of about 3.94 GPa, (ii) an Ultimate Strength of about 139 MPa, (iii) an Extensibility of about 0.818 mm/mm, (iv) a Toughness of about 66.7 MPa and (v) any combination of (i)-(iv).

2. The polypeptide of claim 1, wherein the polypeptide comprises a sequence that is at least 95% identical to a sequence selected from the group consisting of SEQ ID NO:3, 5, 73, 75, 77, 79, 81 and 83.

3. The polypeptide of claim 1, wherein the polypeptide comprises a sequence selected from the group consisting of SEQ ID NO:3, 5, 73, 75, 77, 79, 81 and 83.

4. The polypeptide of claim 1, wherein the polypeptide comprises SEQ ID NO:5.

5. The polypeptide of claim 1, wherein the polypeptide consists of SEQ ID NO:5.

6. A substantially purified polypeptide comprising an N-terminal region that is at least 60% identical to SEQ ID NO:7 from amino acid 1 to about 167, followed by a tandem array of 16-20 repeat units of about 200-370 amino acid in length and having a C-terminal domain comprising at least 40% identity to SEQ ID NO:7 from about amino acid 6236 to 6333, wherein the polypeptide has a property selected from the group consisting of (i) a Young's modulus of about 9.8 to 10.4 GPa, (ii) an Ultimate strength of about 636 to 687 MPa, (iii) an Extensibility of about 0.505 to 0.83 mm/mm, (iv) a Toughness of about 230 MPa to 376 MPa and (v) any combination of (i)-(iv).

7. The polypeptide of claim 6, wherein the polypeptide is about 4400-6300 amino acids in length and about 430 to about 630 kiloDaltons in molecular weight.

8. The polypeptide of claim 7, wherein the polypeptide is at least 90% identical to SEQ ID NO:7 or 87.

9. The polypeptide of claim 8, wherein the polypeptide is at least 95% identical to SEQ ID NO:7 or 87.

10. The polypeptide of claim 9, wherein the polypeptide comprises SEQ ID NO:7.

11. The polypeptide of claim 9, wherein the polypeptide comprises SEQ ID NO:87.

12. A polynucleotide encoding any one of the polypeptides of claim 1 or 6.

13. A vector comprising the polynucleotide of claim 12.

14. A host cell comprising the polynucleotide of claim 12.

15. A substantially purified polypeptide comprising a sequence selected from the group consisting of SEQ ID NO:3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107.

16. An isolated polynucleotide molecule selected from the group consisting of:
a) a polynucleotide molecule comprising a nucleotide sequence which is at least 80% identical to the nucleotide sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106;
b) a polynucleotide molecule which encodes a polypeptide comprising the amino acid sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107; and
c) a polynucleotide molecule which encodes a naturally occurring allelic variant of a polypeptide comprising the amino acid sequence of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105 and 107, wherein the polynucleotide molecule hybridizes to a polynucleotide molecule comprising SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, or 106, or a complement thereof, under moderate to highly stringent conditions.

17-22. (canceled)

23. A method for producing a silk dragline, the method comprising culturing the recombinant host cell of claim 14 under conditions suitable for expression of the polypeptide, such that the polypeptide is produced.

24. A recombinant silk fiber comprising a polypeptide of any one of claim 1 or 6.

25. A copolymer fiber comprising at least two polypeptides of any of claim 1 or 6.

26. A material made from a polypeptide of claim 23.

27. The material of claim 26, wherein the material is a textile material.

28. The material of claim 26, wherein the material is a biomaterial.
